(19) United States
(12) Patent Application Publication
(10) Pub. No.: US 2005/0152906 A1
(43) Pub. Date: Jul. 14, 2005

(54) Specific human antibodies

(76) Inventors: Avigdor Levanon, Rehovot (IL); Tikva Vogel, Rehovot (IL); Daniel Plaksin, Rehovot (IL); Tuvia Peretz, Hod Hasharon (IL); Boaz Amit, Kiron (IL); Lena Cooperman, Rishon Lezion (IL); Yocheved Hagay, Rehovot (IL); Esther Szanton, Rehovot (IL); Yariv Kanfi, Petach Tikva (IL); Rachel Ben-Levy, Bnei Reem (IL); Tali Szrajber, Petach-Tikva (IL)

Correspondence Address: Deborah A. Somerville;KENYON & KENYON, One Broadway, New York, NY, 10004, US

(21) Appl. No.: 10/881,405

(22) Filed: Jun. 30, 2004

Related U.S. Application Data
(60) Provisional application No. 60/484,235, filed on Jun. 30, 2003.

(51) Int. Cl. A61K 39/395; A61K 31/7072; A61K 31/60; A61K 31/522; C07K 16/30

(52) U.S. Cl. 424/155.1; 530/388.22; 530/388.8; 514/49; 514/165; 514/263.31

(57) ABSTRACT

The present invention provides antibodies that bind an epitope of PSGL-1 comprising the motif D-X-Y-D, wherein X represents any amino acid or the covalent linkage between D and Y, and Y is sulfated, which antibody can be complexed with one or more copies of an agent. The antibodies of the invention can be used in a method of inducing antibody-dependent cell cytotoxicity and/or stimulating natural killer (NK) cells or T cells. In addition, by administering these antibodies to a patient in need thereof, a method of inducing cell death is provided. A method of preventing infection by a virus (e.g., HIV) by administering to a patient in need thereof an antibody of the present invention is also provided. The, present invention also provides a method of introducing an agent into a cell that expresses sulfated PSGL-1 by coupling or complexing an agent to an antibody of the present invention and administering the complex to the cell. Finally, the present invention provides methods of diagnosis, prognosis and staging using the present antibodies.

### FIELD OF THE INVENTION

The present invention relates to antibodies that bind to particular epitopes that are present on cells, such as cancer cells, metastatic cells, leukemia cells, leukocytes, and platelets, and that are important in such diverse physiological phenomena as cell rolling, metastasis, inflammation, and auto-immune diseases. More particularly, the antibodies may have anti-cancer activity, anti-metastatic activity, anti-leukemia activity, anti-viral activity, anti-infection activity, and/or activity against other diseases, such as inflammatory diseases, autoimmune diseases, viral infection, cardiovascular diseases such as myocardial infarction, retinopathic diseases, and diseases caused by sulfated tyrosine-dependent protein-protein interactions. In addition, the antibodies of the present invention may be used as a targeting agent to direct a therapeutic to a specific cell or site within the body.

### BACKGROUND OF THE INVENTION

Leukemia, lymphoma, and myeloma are cancers that originate in the bone marrow and lymphatic tissues and are involved in uncontrolled growth of cells. Acute lymphoblastic leukemia (ALL) is a heterogeneous disease that is defined by specific clinical and immunological characteristics. Like other forms of ALL, the definitive cause of most cases of B cell ALL (B-ALL) is not known; although, in many cases, the disease results from acquired genetic alterations in the DNA of a single cell, causing abnormalities and continuous multiplication. Prognosis for patients afflicted with B-ALL is significantly worse than for patients with other leukemias, both in children and in adults. Chronic lymphocytic leukemia (CLL), one example of which is B cell CLL (B-CLL), is a slowly progressing form of leukemia, characterized by an increased number of lymphocytes. Acute myelogenous leukemia (AML) is a heterogeneous group of neoplasms having a progenitor cell that, under normal conditions, gives rise to terminally differentiated cells of the myeloid series (erythrocytes, granulocytes, monocytes, and platelets). As in other forms of neoplasia, AML is associated with acquired genetic alterations that result in replacement of normally differentiated myeloid cells with relatively undifferentiated blasts, exhibiting one or more type of early myeloid differentiation. AML generally evolves in the bone marrow and, to a lesser degree, in the secondary hematopoietic organs. Primarily, AML affects adults and peaks in incidence between the ages of 15-40, but it is also known to affect both children and older adults. Nearly all patients with AML require treatment immediately after diagnosis to achieve clinical remission, in which there is no evidence of abnormal levels of circulating undifferentiated blast cells.

Ligand for Isolated scFv Antibody Molecules

Platelets, fibrinogen, GPIb, selecting, and PSGL-1 (P-Selectin Glycoprotein Ligand-1) each play an important role in several pathogenic conditions or disease states, such as abnormal or pathogenic inflammation, abnormal or pathogenic immune reactions, autoimmune reactions, metastasis, abnormal or pathogenic adhesion, thrombosis and/or restenosis, and abnormal or pathogenic aggregation. Thus, antibodies that bind to or cross-react with platelets and with these molecules would be useful in the diagnosis and treatment of diseases and disorders involving these and other pathogenic conditions.

Platelets

Platelets are well-characterized components of the blood system and play several important roles in hemostasis, thrombosis and/or restenosis. Damage to blood vessel sets in motion a process known as hemostasis, which is characterized by a series of sequential events. The initial reaction to damaged blood vessels is the adhesion of platelets to the affected region on the inner surface of the vessel. The next step is the aggregation of many layers of platelets onto the previously adhered platelets, forming a hemostatic plug and sealing the vessel wall. The hemostatic plug is further strengthened by the deposition of fibrin polymers. The clot or plug is degraded only when the damage has been repaired.

Circulating platelets are cytoplasmic particles released from the periphery of megakaryocytes. Platelets play an important role in hemostasis. Upon vascular injury, platelets adhere to damaged tissue surfaces and attach to one another (cohesion). This sequence of events occurs rapidly, forming a structureless mass (commonly called a platelet plug or thrombus) at the site of vascular injury. The cohesion phenomenon, also known as aggregation, may be initiated in vitro by a variety of substances, or agonists, such as collagen, adenosine-diphosphate (ADP), epinephrine, serotonin, and ristocetin. Aggregation is one of the numerous in vitro tests performed as a measure of platelet function.

Importance of Platelets in Metastasis

Tumor metastasis is perhaps the most important factor limiting the survival of cancer patients. Accumulated data indicate that the ability of tumor cells to interact with host platelets represents one of the indispensable determinants of metastasis (Oleksowicz, Thrombosis Res. 79: 261-74 (1995)). When metastatic cancer cells enter the blood stream, multicellular complexes composed of platelets and leukocytes coating the tumor cells are formed. These complexes, which may be referred to as microemboli, aid the tumor cells in evading the immune system. The coating of tumor cells by platelets requires expression of P-selectin by the platelets.

It has been demonstrated that the ability of tumor cells to aggregate platelets correlates with the tumor cells' metastasis potential, and inhibition of tumor-induced platelet aggregation has been shown to correlate with the suppression of metastasis in rodent models. It has been demonstrated that tumor cell interaction with platelets involves membrane adhesion molecules and agonist secretion. Expression of immuno-related platelet glycoproteins has been identified on tumor cell lines. It was demonstrated that platelet immuno-related glycoproteins, GPIb, GPIIb/IIIa, GPIb/IX and the integrin αᵥ subunit are expressed on the surface of breast tumor cell lines (Oleksowicz, (1995), supra; Kamiyama et al., J. Lab. Clin. Med. 117(3): 209-17 (1991)).

Gasic et al. (PNAS 61:46-52 (1968)) showed that antibody-induced thrombocytopenia markedly reduced the number and volume of metastases produced by CT26 colon adenocarcinoma, Lewis lung carcinoma, and B16 melanoma (Karpatkin et al., J. Clin. Invest. 81(4): 1012-19 (1988); Clezardin et al., Cancer Res. 53(19): 4695-700 (1993)). Furthermore, a single polypeptide chain (60 kd) was found to be expressed on surface membrane of HEL cells that is closely related to GPIb and corresponds to an incompletely or abnormally O-glycosylated GPIbα subunit (Kieffer et al., J. Biol. Chem. 261(34): 15854-62 (1986)).

GPIb Complex

Each step in the process of hemostasis requires the presence of receptors on the platelet surface. One receptor that is important in hemostasis is the glycoprotein Ib-IX complex (also known as CD42). This receptor mediates adhesion (initial attachment) of platelets to the blood vessel wall at sites of injury by binding von Willebrand factor (vWF) in the subendothelium. It also has crucial roles in two other platelet functions important in hemostasis: (a) aggregation of platelets induced by high shear in regions of arterial stenosis and (b) platelet activation induced by low concentrations of thrombin.

The GPIb-IX complex is one of the major components of the outer surface of the platelet plasma membrane. This complex comprises three membrane-spanning polypeptides—a disulfide-linked 130 kDa α-chain and 25 kDa β-chain of GPIb and a noncovalently associated GPIX (22 kDa). All of the subunits are presented in equimolar amounts on the platelet membrane for efficient cell-surface expression and function of CD42 complex, indicating that proper assembly of the three subunits into a complex is required for full expression on the plasma membrane. The α-chain of GPIb consists of three distinct structural domains: (1) a globular N-terminal peptide domain containing leucine-rich repeat sequences and Cys-bonded flanking sequences; (2) a highly glycosylated mucin-like macroglycopeptide domain; and (3) a membrane-associated C-terminal region that contains the disulfide bridge to GPIbα and transmembrane and cytoplasmic sequences.

Several lines of evidence indicate that the vWF and thrombin-binding domain of the GPIb-IX complex reside in a globular region encompassing approximately 300 amino acids at the amino terminus of GPIbα. As human platelet GPIb-IX complex is a key membrane receptor mediating both platelet function and reactivity, recognition of subendothelial-bound vWF by GPIb allows platelets to adhere to damaged blood vessels. Further, binding of vWF to GPIbα also induces platelet activation, which may involve the interaction of a cytoplasmic domain of the GPIb-IX with cytoskeleton or phospolipase A2. Moreover, GPIbα contains a high-affinity binding site for α-thrombin, which facilitates platelet activation by an as-yet poorly defined mechanism.

The N-terminal globular domain of GPIbα contains a cluster of negatively charged amino acids. Several lines of evidence indicate that in transfected CHO cells expressing GPIb-IX complex and in platelet GPIbα, the three tyrosine residues contained in this domain (Tyr-276, Tyr-278, and Tyr-279) undergo sulfation.

Protein Sulfation

Protein sulfation is a widespread post-translational modification that involves enzymatic covalent attachment of sulfate, either to sugar side chains or to the polypeptide backbone. This modification occurs in the trans-Golgi compartment. Sulfated proteins include secretory proteins, proteins targeted for granules, and the extracellular regions of plasma membrane proteins. Tyrosine is an amino acid residue presently known to undergo sulfation. Kehoe et al., Chem. Biol. 7: R57-61 (2000). Other amino acids, e.g., threonine, may also undergo sulfation, particularly in diseased cells.

A number of proteins have been found to be tyrosine-sulfated, but the presence of three or more sulfated tyrosines in a single polypeptide, as was found on GPhb, is not common. GPIbα (CD42), which is expressed by platelets and megakaryocytes and mediates platelet attachment to and rolling on subendothelium via binding with vWF, also contains numerous negative charges at its N-terminal domain. Such a highly acidic and hydrophilic environment is thought to be a prerequisite for sulfation, because tyrosylprotein sulfotransferase specifically recognizes and sulfates tyrosines adjacent to acidic amino residues (Bundgaard et al., J. Biol. Chem. 272:21700-05 (1997)). Full sulfation of the acidic region of GPIbα yields a region with a remarkable negative charge density—13 negative charges within a 19 amino acid stretch—and is a candidate site for electrostatic interaction with other proteins.

It is also thought that sulfated N-terminal tyrosines influence the role of CC-chemokine receptors, such as CCR5, which serve as co-receptors with related seven transmembered segment (7TMS) receptor for entry of human and simian immunodeficiency viruses (HIV-1, HIV-2, and SIV) into target cells. For example, it is thought that sulfated N-terminal tyrosines contribute to the binding of CCR5 to MIP-1α, MIP-1β, and HIV-1 gp120/CD4 complexes and to the ability of HIV-1 to enter cells expressing CCR5 and CD4. CXCR4, another important HIV-1 co-receptor, is also sulfated (Farzan et al., Cell 96(5): 667-76 (1999)). Tyrosine sulfation plays a less significant role in CXCR4-dependent HIV-1 entry than CCR5-dependent entry; thus demonstrating a possible role for tyrosine sulfation in the CXC-chemokine family and underscoring a general difference in HIV-1 utilization of CCR5 and CXCR4 (Farzan et al., J. Biol. Chem. 277(33): 29,484-89 (2002)).

Selectins and PSGL-1

The P-, E-, and L-Selectins are members of a family of adhesion molecules that, among other functions, mediate rolling of leukocytes on vascular endothelium. P-Selectin is stored as granules in platelets and is transported to the surface after activation by thrombin, histamine, phorbol ester, or other stimulatory molecules. P-Selectin is also expressed on activated endothelial cells. E-Selectin is expressed on endothelial cells, and L-Selectin is expressed on neutrophils, monocytes, T cells, and B cells.

PSGL-1 (also called CD162) is a mucin glycoprotein ligand for P-Selectin, E-Selectin, and L-Selectin that shares structural similarity with GPIb (Afshar-Kharghan et al. (2001), supra). PSGL-1 is a disulfide-linked homodimer that has a PACE (Paired Basic Amino Acid Converting Enzymes) cleavage site. PSGL-1 also has three potential tyrosine sulfation sites followed by 10-16 decamer repeats that are high in proline, serine, and threonine. The extracellular portion of PSGL-1 contains three N-linked glycosylation sites and has numerous sialylated, fucosylated O-linked oligosaccharide branches (Moore et al., J. Biol. Chem. 118: 445-56 (1992)). Most of the N-glycan sites and many of the O-glycan sites are occupied. The structures of the O-glycans of PSGL-1 from human HL-60 cells have been determined. Subsets of these O-glycans are core-2, sialylated and fucosylated structures that are required for binding to selectins. Tyrosine sulfation of an amino-terminal region of PSGL-1 is also required for binding to P-Selectin and L-Selectin. Further, there is an N-terminal propeptide that is probably cleaved post-translationally.

PSGL-1 has 361 residues in HL60 cells, with a 267 residue extracellular region, 25 residue trans-membrane region, and a 69 residue intracellular region, and forms a disulfide-bonded homodimer or heterodimer on the cell surface (Afshar-Kharghan et al., Blood 97: 3306-12 (2001)). The sequence encoding PSGL-1 is in a single exon, so alternative splicing should not be possible. However, PSGL-1 in HL60 cells, and in most cell lines, has 15 consecutive repeats of a 10 residue consensus sequences present in the extracellular region, although there are 14 and 16 repeats of this sequence in polymorphonuclear leukocytes, monocytes, and several other cell lines, including most native leukocytes.

PSGL-1 is expressed on neutrophils as a dimer, with apparent molecular weights of both 250 kDa and 160 kDa, whereas on HL60 the dimeric form is approximately 220 kDa. When analyzed under reducing conditions, each subunit is reduced by half. Differences in molecular mass may be due to polymorphisms in the molecule caused by the presence of different numbers of decamer repeats (Leukocyte Typing VI. Edited by T. Kishimoto et al. (1997)).

Most blood leukocytes, such as neutrophils, monocytes, leukocytes, subset of B cells, and all T cells express PSGL-1 (Kishimoto et al. (1997), supra). PSGL-1 mediates rolling of leukocytes on activated endothelium, on activated platelets, and on other leukocytes and inflammatory sites and mediates rolling of neutrophils on P-Selectin. PSGL-1 may also mediate neutrophil-neutrophil interactions via binding with L-Selectin, thereby mediating inflammation (Snapp et al., Blood 91(1): 154-64 (1998)).

Leukocyte rolling is important in inflammation, and interaction between P-Selectin (expressed by activated endothelium and on platelets, which may be immobilized at sites of injury) and PSGL-1 is instrumental for tethering and rolling of leukocytes on vessel walls (Ramachandran et al., PNAS 98(18): 10166-71 (2001); Afshar-Kharghan et al. (2001), supra). Cell rolling is also important in metastasis, and P- and E-Selectin on endothelial cells is believed to bind metastatic cells, thereby facilitating extravasation from the blood stream into the surrounding tissues.

Thus, PSGL-1 has been found on all leukocytes: neutrophils, monocytes, lymphocytes, activated peripheral T cells, granulocytes, eosinophils, platelets and on some CD34 positive stem cells and certain subsets of B cells. P-Selectin is selectively expressed on activated platelets and endothelial cells. Interaction between P-Selectin and PSGL-1 promotes rolling of leukocytes on vessel walls, and abnormal accumulation of leukocytes at vascular sites results in various pathological inflammations. Stereo-specific contributions of individual tyrosine sulfates on PSGL-1 are important for the binding of P-Selectin to PSGL-1. Charge is also important for binding: reducing NaCl (from 150 to 50 mM) enhanced binding (Kd˜75 nM). Tyrosine-sulfation on PSGL-1 enhances, but is not ultimately required for PSGL-1 adhesion on P-Selectin. PSGL-1 tyrosine sulfation supports slower rolling adhesion at all shear rates and supports rolling adhesion at much higher shear rates (Rodgers et al., Biophys. J. 81: 2001-09 (2001)). Moreover, it has been suggested that PSGL-1 expression on platelets is 25-100 fold lower than that of leukocytes (Frenette et al., J. Exp. Med. 191(8): 1413-22 (2000)).

A commercially available monoclonal antibody to human PSGL-1, KPL1, has been shown to inhibit the interactions between PSGL-1 and P-selectin and between PSGL-1 and L-selectin. The KPL1 epitope was mapped to the tyrosine sulfation region of PSGL-1 (YEYLDYD) (SEQ ID NO:1) (Snapp et al., Blood 91(1):154-64 (1998)).

Pretreatment of tumor cells with O-sialoglycoprotease, which removes sialylated, fucosylated mucin ligands, also inhibited tumor cell-platelet complex formation. In vivo experiments indicate that either of these treatments results in greater monocyte association with circulating tumor cells, suggesting that reducing platelet binding increases access by immune cells to circulating tumor cells (Varki and Varki, Braz. J. Biol. Res. 34(6): 711-17 (2001)).

Fibrinogen

There are two forms of normal human fibrinogen—normal (γ) and γ prime, each of which is found in normal individuals. Normal fibrinogen, which is the more abundant form (approximately 90% of the total fibrinogen found in the body), is composed of two identical 55 kDa α chains, two identical 95 kDa β chains, and two identical 49.5 kDa γ chains. Normal variant fibrinogen, which is the less abundant form (approximately 10% of the fibrinogen found in the body), is composed of two identical 55 kDa α chains, two identical 95 kDa β chains, one 49.5 kDa γ chain, and one variant 50.5 kDa γ prime chain. The gamma and gamma prime chains are both coded for by the same gene, with alternative splicing occurring at the 340 end. Normal gamma chain is composed of amino acids 1-411 and normal variant gamma prime chain is composed of 427 amino acids, of which amino acids 1-407 are the same as those in the normal gamma chain and amino acids 408-427 are VRPEHPAETEYDSLYPEDDL (SEQ ID NO:2). This region is normally occupied with thrombin molecules.

Fibrinogen is converted into fibrin by the action of thrombin in the presence of ionized calcium to produce coagulation of the blood. Fibrin is also a component of thrombi, and acute inflammatory exudates.

Therefore, an object of the invention is to provide various antibodies or polypeptides that bind sulfated PSGL-1 and methods of use thereof.

Specifically, an object of the invention is to provide methods of activating ADCC or stimulating natural killer (NK) or T cells by administering the antibodies of the present invention.

Another specific object of the invention is to provide a method of inducing cell death.

Yet another specific object of the invention is to provide a method of preventing infection by a virus, such as HIV, comprising administering to a patient in need thereof an antibody as herein.

Another specific object of the invention is to provide a method of introducing an agent into a cell that expresses sulfated PSGL-1 having the following steps: coupling or complexing the agent to an antibody as described herein and administering the antibody-agent couple or complex to the cell is provided

Finally, it is a specific objective of the present invention to provide methods of diagnosis, prognosis, and staging using the present antibodies.

### SUMMARY OF THE INVENTION

The present invention provides antibodies or polypeptides that bind an epitope of PSGL-1 comprising the motif D-X-Y-D (SEQ ID NO:3), wherein X represents any amino acid or the covalent linkage between D and Y, and Y is sulfated, which antibody can be coupled to or complexed with multiple copies of an agent selected from the group consisting of anti-cancer, anti-leukemic, anti-metastasis, anti-neoplastic, anti-disease, anti-adhesion, anti-thrombosis, anti-restenosis, anti-autoimmune, anti-aggregation, anti-bacterial, anti-viral, and anti-inflammatory agents.

The antibodies of the invention can be used in a method of inducing antibody-dependent cell cytotoxicity and/or stimulating natural killer (NK) cells or T cells. In addition, by administering these antibodies to a patient in need thereof, a method of inducing cell death is provided. A method of preventing infection by a virus (e.g., HIV) by administering to a patient in need thereof an antibody of the present invention is also provided. The present invention also provides a method of introducing an agent into a cell that expresses sulfated PSGL-1 by coupling or complexing an agent to an antibody of the present invention and administering the antibody-agent couple or complex to the cell. The present invention further provides a method of identifying, isolating and purifying tumor cell markers. Finally, the present invention provides methods of diagnosis, prognosis and staging using the present antibodies.

### BRIEF DESCRIPTION OF THE DRAWINGS

The invention is herein described in more detail, by way of example only, and not by way of limitation, with reference to the accompanying drawings described below, wherein:

FIG. 1 shows a Western blot of partially purified AML-R1 cell lysate before and after passage through Y1-IgG affinity column.

FIG. 2 shows that, of three tyrosines in the purified protein's sulfated-tyrosine motif, tyrosines 2 and 3 are sulfated.

FIG. 3 shows Y1-IgG (20 μg/ml) mediated ADCC (percent cytotoxicity) in primary B-CLL samples.

FIG. 4 shows Y1-IgG-mediated ADCC (percent cytotoxicity) by PBMC against AML cells.

FIG. 5 shows increaseed ADCC (percent cytotoxicity) in ML-2 cells as a function of Y1-IgG concentration.

FIG. 6 shows ADCC (percent cytotoxicity) by PBMC against ML-2 as a function of competition between Y1-IgG and KPL-1.

FIG. 7A shows analysis of Y1-IgG-mediated ADCC (percent cytotoxicity) by natural killer cells from normal donors and B-CLL patients against ML2 cells. FIG. 7B shows the involvement of CD14+ cells (monocytes) in ADCC against M12 targets.

FIG. 8 shows expression of CD69 (an early activation marker) on NK cells mediated by Y1.

FIG. 9 shows apoptotic effect of Y1-IgG on mononuclaer cells (CD19+, CD5+) from B-CLL patients by FACS analysis.

FIG. 10 shows analysis of ADCC activity (percent cytotoxicity) against mononuclear cells from human B-CLL patients, i.e., primary human B-CLL cells (KBC115 and KBC116 cells) mediated by Y1-IgG and Rituximab.

FIG. 11 shows analysis of CDC activity (percent lysis) against mononuclear cells from human B-CLL patients (KBC156, KBC159, KBC160, KBC166, and RAJI cells) mediated by Y1-IgG, Rituximab, and Campath® in the presence and absence of patient plasma.

FIG. 12 shows reaction scheme for preparation of antibody linked to morpholino-doxorubicin.

FIG. 13 shows cytotoxicity of an antibody-agent complex, namely Y1-morpholinodaunorubicin (Y1-M-DNR) and Y1-morpholinodoxorubicin (Y1-M-Dox) complexes in cord blood and AML cells.

FIG. 14 shows cytotoxicity of the antibody-agent complex Y1-M-DNR against 2 patient AML samples (M4 and M5 stage) and against CD34+ cells.

FIG. 15 shows cytotoxicity of various Y1 complexes as a percent of control in B-ALL cells.

FIG. 16A shows binding of Y1 scFv to KU812 cells and FIG. 16B shows the surface expression of GPIb on sulfate starved KU812 cells.

FIG. 17A shows inhibitory effects of sulfated peptides DLYDYYPE on the binding of Y1-scFv to platelets. FIG. 17B shows the effects of substitution mutant peptides in Y1-scFv platelet binding assay.

FIG. 18A shows effects of mutant peptides in the inhibition of Y1-scFv binding to purified glycocalicin. FIG. 18B shows the binding of Y1scFv to peptides covalently coupled to CovaLink™ Plates by ELISA.

FIG. 19 shows binding of Y1 to immobilized, sulfated peptides derived from PSGL-1.

FIG. 20 shows percent activity of Y1 binding to non-sulfated PSGL-1 and to PSGL-1 sulfated in the first, second, and third positions.

FIG. 21 some potential Y1 binding motifs that are highly acidic and have sulfated tyrosines.

FIG. 22 shows recognition of small cell lung carcinoma (SCLC) lysate by Y1.

FIG. 23 shows endocytosis of Y1 into primary AML cells.

FIG. 24 shows endocytosis of Y1 into primary AML cells.

FIG. 25 shows analysis of Y1 binding to healthy CD34+stem cells.

FIG. 26 shows analysis of Y1 binding to healthy CD34+stem cells.

FIG. 27 shows internalization of Y1 into primary AML cells at 37° C.

FIG. 28 shows visualization of Y1 staining in primary AML cells after stripping membrane-bound protein by acid treatment.

FIG. 29 shows visualization of Y1 staining in primary AML cells after stripping membrane-bound protein by pronase treatment.

FIG. 30 shows visualization of Y1 staining in primary AML cells after acid treatment or sucrose pre-incubation at 4° C. (FIG. 30A) and at 37° C. (FIG. 30B).

FIG. 31 shows that Y1-scFv effectively inhibits binding of activated human platelets to ML2 cells.

FIG. 32 shows the effect of Y1-scFv (10 μg/ml) on ML2 cell rolling on immobilized rh-P-Selectin at low density (0.2 μg/ml).

FIG. 33 shows the effect of Y1-scFv (10 μg/ml) on ML2 cell rolling on immobilized rh-P-Selectin at high density (1.0 μg/ml).

FIG. 34 shows the effect of Y1-IgG (1 μg/ml) on ML2 cell rolling on immobilized rh-P-Selectin (1.0 μg/ml) at various shear stress forces.

FIG. 35 shows the effect of increasing concentrations of Y1-scFv on human neutrophil rolling on immobilized rh-P-Selectin at high density (1.0 μg/ml).

FIG. 36 shows the effect of Y1-IgG on human neutrophil rolling on immobilized rh-P-Selectin at high density (1.0 μg/ml).

### DETAILED DESCRIPTION OF THE INVENTION

Antibodies (Abs), or immunoglobulins (Igs), are protein molecules that bind to antigen. Each functional binding unit of naturally occurring antibodies is composed of units of four polypeptide chains (2 heavy and 2 light) linked together by disulfide bonds. Each of the chains has a constant and variable region. Naturally occurring antibodies can be divided into several classes including IgG, IgM, IgA, IgD, and IgE, based on their heavy chain component. The IgG class encompasses several sub-classes including, but not restricted to, IgG₁, IgG₂, IgG₃, and IgG₄. Immunoglobulins are produced in vivo by B lymphocytes, and each such molecule recognizes a particular foreign antigenic determinant and facilitates clearing of that antigen.

Antibodies may be produced and used in many forms, including antibody complexes. As used herein, the term “antibody complex” or “antibody complexes” is used to mean a complex of one or more antibodies with another antibody or with an antibody fragment or fragments, or a complex of two or more antibody fragments. Examples of antibody fragments include Fv, Fab, F(ab′)₂, Fc, and Fd fragments. Therefore, an antibody according to the present invention encompasses a complex of an antibody or fragment thereof.

As used herein in the specification and in the claims, an Fv is defined as a molecule that is made up of a variable region of a heavy chain of a human antibody and a variable region of a light chain of a human antibody, which may be the same or different, and in which the variable region of the heavy chain is connected, linked, fused, or covalently attached to, or associated with, the variable region of the light chain. The Fv can be a single chain Fv (scFv) or a disulfide stabilized Fv (dsFv). An scFv is comprised of the variable domains of each of the heavy and light chains of an antibody, linked by a flexible amino-acid polypeptide spacer, or linker. The linker may be branched or unbranched. Preferably, the linker is 0-15 amino acid residues, and most preferably the linker is (Gly₄Ser)₃.

The Fv molecule, itself, is comprised of a first chain and a second chain, each chain having a first, second and third hypervariable region. The hypervariable loops within the variable domains of the light and heavy chains are termed Complementary Determining Regions (CDRs). There are CDR1, CDR2, and CDR3 regions in each of the heavy and light chains. These regions are believed to form the antigen binding site and can be specifically modified to yield enhanced binding activity. The most variable of these regions in nature is the CDR3 region of the heavy chain. The CDR3 region is understood to be the most exposed region of the Ig molecule and, as shown and provided herein, is the site primarily responsible for the selective and/or specific binding characteristics observed.

A fragment of an Fv molecule is defined as any molecule smaller than the original Fv that still retains the selective and/or specific binding characteristics of the original Fv. Examples of such fragments include but are limited to (1) a minibody, which comprises a fragment of the heavy chain only of the Fv, (2) a microbody, which comprises a small fractional unit of antibody heavy chain variable region (International Application No. PCT/IL99/00581), (3) similar bodies having a fragment of the light chain, and (4) similar bodies having a functional unit of a light chain variable region. It should be appreciated that a fragment of an Fv molecule can be a substantially circular or looped polypeptide.

As used herein the term “Fab fragment” is a monovalent antigen-binding fragment of an immunoglobulin. A Fab fragment is composed of the light chain and part of the heavy chain.

An F(ab′)₂ fragment is a bivalent antigen binding fragment of an immunoglobulin obtained by pepsin digestion. It contains both light chains and part of both heavy chains.

An Fc fragment is a non-antigen-binding portion of an immunoglobulin. It contains the carboxy-terminal portion of heavy chains and the binding sites for the Fc receptor.

An Fd fragment is the variable region and first constant region of the heavy chain of an immunoglobulin.

Polyclonal antibodies are the product of an immune response and are formed by a number of different B lymphocytes. Monoclonal antibodies are derived from one clonal B cell.

A cassette, as applied to polypeptides and as defined in the present invention, refers to a given sequence of consecutive amino acids that serves as a framework and is considered a single unit and is manipulated as such. Amino acids can be replaced, inserted into, removed, or attached at one or both ends. Likewise, stretches of amino acids can be replaced, inserted into, removed, or attached at one or both ends.

The term “epitope” is used herein to mean the antigenic determinant or recognition site or antigen site that interacts with an antibody, antibody fragment, antibody complex or a complex having a binding fragment thereof or T cell receptor. The term epitope is used interchangeably herein with the terms ligand, domain, and binding region.

Selectivity is herein defined as the ability of a targeting molecule to choose and bind one entity or cell state from a mixture of entities or entity states, all entities or entity states of which may be specific for the targeting molecule.

The term “affinity” as used herein is a measure of the binding strength (association constant) between a binding molecule (e.g., one binding site on an antibody) and a ligand (e.g., antigenic determinant). The strength of the sum total of noncovalent interactions between a single antigen-binding site on an antibody and a single epitope is the affinity of the antibody for that epitope. Low affinity antibodies bind antigen weakly and tend to dissociate readily, whereas high-affinity antibodies bind antigen more tightly and remain bound longer. The term “avidity” differs from affinity, because the former reflects the valence of the antigen-antibody interaction.

Specificity of antibody-antigen interaction: Although the antigen-antibody reaction is specific, in some cases antibodies elicited by one antigen can cross-react with another unrelated antigen. Such cross-reactions occur if two different antigens share a homologous or similar structure, epitope, or an anchor region thereof, or if antibodies specific for one epitope bind to an unrelated epitope possessing similar structure conformation or chemical properties.

A platelet is a disc-like cytoplasmic fragment of a megakaryocyte that is shed in the marrow sinus and subsequently circulates in the peripheral blood stream. Platelets have several physiological functions including a major role in clotting. A platelet contains centrally located granules and peripheral clear protoplasm, but has no definite nucleus.

Agglutination as used herein means the process by which suspended bacteria, cells, discs, or other particles of similar size are caused to adhere and form into clumps. The process is similar to precipitation but the particles are larger and are in suspension rather than being in solution.

The term aggregation means a clumping of platelets induced in vitro, and thrombin and collagen, as part of a sequential mechanism leading to the formation of a thrombus or hemostatic plug.

Conservative amino acid substitution is defined as a change in the amino acid composition by way of changing one or two amino acids of a peptide, polypeptide or protein, or fragment thereof. The substitution is of amino acids with generally similar properties (e.g., acidic, basic, aromatic, size, positively or negatively charged, polarity, non-polarity) such that the substitutions do not substantially alter peptide, polypeptide or protein characteristics (e.g., charge, isoelectric point, affinity, avidity, conformation, solubility) or activity. Typical substitutions that may be performed for such conservative amino acid substitution may be among the groups of amino acids as follows:
glycine (G), alanine (A), valine (V), leucine (L) and isoleucine (I)
aspartic acid (D) and glutamic acid (E)
alanine (A), serine (S) and threonine (T)
histidine (H), lysine (K) and arginine (R)
asparagine (N) and glutamine (Q)
phenylalanine (F), tyrosine (Y) and tryptophan (W)

Conservative amino acid substitutions can be made in, e.g., regions flanking the hypervariable regions primarily responsible for the selective and/or specific binding characteristics of the molecule, as well as other parts of the molecule, e.g., variable heavy chain cassette. Additionally or alternatively, modification can be accomplished by reconstructing the molecules to form full-size antibodies, diabodies (dimers), triabodies (timers), and/or tetrabodies (tetramers) or to form minibodies or microbodies.

A phagemid is defined as a phage particle that carries plasmid DNA. Phagemids are plasmid vectors designed to contain an origin of replication from a filamentous phage, such as m13 of fd. Since it carries plasmid DNA, the phagemid particle does not have sufficient space to contain the full complement of the phage genome. The component that is missing from the phage genome is information essential for packaging the phage particle. In order to propagate the phage, therefore, it is necessary to culture the desired phage particles together with a helper phage strain that complements the missing packaging information.

A promoter is a region on DNA at which RNA polymerase binds and initiates transcription.

A phage display library (also termed phage peptide/antibody library, phage library, or peptide/antibody library) comprises a large population of phages (10⁸ or larger), each phage particle displaying a different peptide or polypeptide sequence. These peptide or polypeptide fragments may constructed to be of variable length. The displayed peptide or polypeptide can be derived from, but need not be limited to, human antibody heavy or light chains.

A pharmaceutical composition refers to a formulation which comprises an antibody or peptide or polypeptide of the invention and a pharmaceutically acceptable carrier, excipient or diluent thereof, or an antibody-pharmaceutical agent (antibody-agent) complex and a pharmaceutically acceptable carrier, excipient or diluent thereof.

An agent in the context of the present invention is useful in the treatment of active disease, prophylactic treatment, or diagnosis of a mammal including, but not restricted to, a human, bovine, equine, porcine, murine, canine, feline, or any other warm-blooded animal. The agent is selected from the group of radioisotope, toxin, oligonucleotide, recombinant protein, antibody fragment, anti-cancer agents, anti-leukemic, anti-metastasis, anti-neoplastic, anti-disease, anti-adhesion, anti-thrombosis, anti-restenosis, anti-autoimmune, anti-aggregation, anti-bacterial, anti-viral, and anti-inflammatory agents. Other examples of such agents include, but are not limited to anti-viral agents including acyclovir, ganciclovir, and zidovudine; anti-thrombosis/restenosis agents including cilostazol, dalteparin sodium, reviparin sodium, and aspirin; anti-inflammatory agents including zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid; anti-autoimmune agents including leflunomide, denileukin diftitox, subreum, WinRho SDF, defibrotide, and cyclophosphamide; and anti-adhesion/anti-aggregation agents including limaprost, clorcromene, and hyaluronic acid.

An anti-leukemia agent is an agent with anti-leukemia activity. For example, anti-leukemia agents include agents that inhibit or halt the growth of leukemic or immature pre-leukemic cells, agents that kill leukemic or pre-leukemic cells, agents that increase the susceptibility of leukemic or pre-leukemic cells to other anti-leukemia agents, and agents that inhibit metastasis of leukemic cells. In the present invention, an anti-leukemia agent may also be an agent with anti-angiogenic activity that prevents, inhibits, retards or halts vascularization of tumors.

The expression pattern of a gene can be studied by analyzing the amount of gene product produced under various conditions, at specific times, in various tissues, etc. A gene is considered to be “over-expressed” when the amount of gene product is higher than that found in a normal control, e.g., non-diseased control.

A given cell may express on its surface a protein having a binding site (or epitope) for a given antibody, but that binding site may exist in a cryptic form (e.g., be sterically hindered or blocked, or lack features needed for binding by the antibody) in the cell in a state, which may be called a first stage (stage I). Stage I may be, e.g., a normal, healthy, non-diseased status. When the epitope exists in cryptic form, it is not recognized by the given antibody, i.e., there is no binding of the antibody to this epitope or to the given cell at stage I. However, the epitope may be exposed by, e.g., undergoing modifications itself, or being unblocked because nearby or associated molecules are modified or because a region undergoes a conformational change. Examples of modifications include changes in folding, changes in post-translational modifications, changes in phospholipidation, changes in sulfation, changes in glycosylation, and the like. Such modifications may occur when the cell enters a different state, which may be called a second stage (stage II). Examples of second states, or stages, include activation, proliferation, transformation, or in a malignant status. Upon being modified, the epitope may then be exposed, and the antibody may bind.

Peptido-mimetics (peptide mimetics) are molecules (e.g., antibodies) that no longer contain any peptide bonds, i.e., amide bonds, between amino acids; however, in the context of the present invention, the term peptide mimetic is intended to include molecules that are no longer completely peptidic in nature, such as pseudo-peptides, semi-peptides and peptoids. Whether completely or partially non-peptide, peptidomimetics according to this invention provide a spatial arrangement of reactive chemical moieties that closely resembles the three-dimensional arrangement of active groups in the peptide on which the peptidomimetic is based. These molecules include small molecules, lipids, polysaccharides, or conjugates thereof.

Phagemids are plasmid vectors designed to contain an origin of replication from a filamentous phage, such as M13 or fd.

A wide spectrum of diseases exists that involves diseased, altered, or otherwise modified cells that express cell-specific and/or disease-specific ligands on their surfaces. These ligands can be utilized to effect recognition, selection, diagnosis and treatment of specific diseases through recognition, selection, diagnosis and treatment of each individual cell. The subject invention provides for peptides or polypeptides that comprise an Fv molecule, a construct thereof, a fragment thereof, a construct of a fragment thereof, or a fragment of a construct, all of which have enhanced binding characteristics. These binding characteristics allow the peptide or polypeptide molecule to bind selectively and/or specifically to a target cell in favor of other cells, the binding specificity and/or selectivity being primarily determined by a first hypervariable region. The Fv can be a scFv or a dsFv.

The Fv molecule described above can be used to target a diseased cell. The diseased cell can be, for example, a cancer cell. Examples of types of cancer that are amenable to diagnosis and/or treatment by specific targeting include, but are not limited to, carcinoma, sarcoma, leukemia, adenoma, lymphoma, myeloma, blastoma, seminoma, and melanoma. Leukemia, lymphoma, and myeloma are cancers that originate in the bone marrow and lymphatic tissues and are involved in uncontrolled growth of cells.

Antibodies that bind to PSGL-1 and/or GPIb were identified using a phage display library and disclosed in U.S. application Ser. Nos. 10/032,423; 10/032,037; 10/029,988; 10/029,926; 09/751,181; 10,189,032; and 60/258,948 and International Application Nos. PCT/US01/49442 and PCT/US01/49440. Specific examples of antibodies disclosed in these applications include the Y1, Y17, and L32 antibodies. These antibodies were isolated from the germ line (DP32) and were discovered to specifically bind to an epitope, found on proteins of the hematopoetic cells, which is sulfated at an N-terminal tyrosine and is thought to be involved in cell migration, e.g. tumor metastasis.

The sulfated epitopes binding to Y1/Y17/L32 are characterized by the presence of sulfated moieties, such as sulfated tyrosine residues or sulfated carbohydrate or lipid moieties, preferably within a cluster of two or more acidic amino acids, which are found on ligands and receptors that play important roles in such diverse processes as inflammation, immune reactions, infection, autoimmune reactions, metastasis, adhesion, thrombosis and/or restenosis, cell rolling, and aggregation. Such epitopes are also found on diseased cells, such as T-ALL cells, B-leukemia cells, B-CLL cells, AML cells, multiple myeloma cells, and metastatic cells. These epitopes are useful targets for the therapeutic mediation of these processes (as well as targeting agents) and for diagnostic procedures.

Moreover, it was found for these antibodies of the present invention that binding is dependent on the stage of development of the cell (AML subtype is classified based on the French-American-British system using the morphology observed under routine processing and cytochemical staining): the antibodies bind to AML cells that are of subtype M3 or above, but not MO or Ml subtype cells. In addition, the antibodies may or may not bind M2 subtype cells. Accordingly, the antibodies of the present invention do not bind normal, healthy bone marrow (e.g., CD34+ cells). It is thought that such differences are based on alterations in PSGL-1 expression and/or sulfation, as well as possible conformational changes in PSGL-1 that expose a slightly different epitope.

In particular, it has been found that KU812 cells, a human chronic myeloid leukemia cell line that expresses low levels of GPIb, binds the Y1 antibody. Following growth of KU812 cells in sodium chlorate, which inhibits sulfation but not expression of the GPIb protein, binding of Y1 to the cells was reduced by 50%. In addition, it has been found that tyrosine-sulfated peptides based on amino acids 273 to 285 of GPIb competitively inhibit binding of the Y1 antibody to platelets, while non-sulfated peptides do not inhibit binding of the Y1 antibody to platelets.

The invention comprises or employs an antibody or fragment thereof that recognizes and binds to an epitope comprising a sulfated tyrosine motif. Such a motif comprises a peptide sequence that is rich in acidic residues (aspartate and glutamate) and contains at least one tyrosine. Recognition and binding depend at least in part on at least one of the tyrosines being sulfated. One such antibody is Y1 or a fragment thereof. Although Y1 is the antibody referred to in the embodiments described herein, this should not be understood as limiting the invention to embodiments that employ Y1. The invention includes embodiments that use other antibodies that bind to an epitope comprising a sulfated tyrosine motif, including but not limited to antibodies related to Y1 and fragments thereof that retain binding specificity.

According to the present invention, provided is an antibody or fragment thereof that binds to an epitope comprising a sulfated tyrosine motif, wherein the binding is dependent on at least one tyrosine of the motif being sulfated. In one embodiment, the antibody mediates antibody-dependent cell cytotoxicity. In a preferred embodiment, the antibody is Y1 or a related antibody, or a fragment thereof.

The invention further provides an agent complexed with (e.g., associated, combined, fused, or linked to) such an antibody or fragment thereof. Between 1 and 16 agent molecules, or more, can be bound to each antibody. The antibody has four disulfide bonds at the hinge region that can be selectively reduced to eight thiol groups. By using a linker that can covalently bond to thiol functions and which carries one agent molecule, up to eight agent molecules can be attached to the antibody. By using a linker that similarly reacts with thiol functions but carries n agent molecules, up to 8n agent molecules can be attached to the antibody. In one embodiment only the heavy chains are complexed with the agent or only the light chains are complexed with the agent, while in a more prefered embodiment, each heavy chain is complexed with about 2 copies of the agent and each light chain is complexed with about 2 copies of the agent.

To those of ordinary skill in the art it is known that an even greater number of agent molecules can be linked to the antibody by using intermediate drug carriers such as natural (e.g. dextran) and synthetic (e.g. HPMA) polymers as well as liposomes (e.g., antibody—linker—carrier—agent). Agents can also be linked directly or indirectly to free amino groups of the antibody. For example, agents can be linked to free ε- or α-amino groups via a linker. Typically an agent is joined to a linker directly, or first to a carrier, which is then joined to a linker. The linker-agent or linker-carrier-agent complex is then joined to the antibody. The antibody-agent complex can be internalized by a tumor cell, wherein the agent brings about the cell's death. In one embodiment, the antibody-agent linkage can be broken inside the cell by, for example, acid cleavage or enyzme cleavage. In a preferred embodiment, the antibody is Y1 or a related antibody, or a fragment thereof.

The invention further provides a composition for treating a disease comprising Y1 or a Y1-agent complex.

In one embodiment, the present invention provides methods of inducing or activating ADCC by administering the antibodies of the present invention. Accordingly, these antibodies may activate ADCC and/or stimulate natural killer (NK) cells (e.g. CD56+), γδ T-cells, and/or monocytes, which may result in cell lysis. Generally, following administration of an antibody comprising an Fc region or portion of the antibody, said antibody binds to an Fc receptor (FcR) on effector cells, for example, NK cells, triggering the release of perforin and granzyme B and/or induction of Fas B expression, which then leads to apoptosis. Binding of FasL expressed on effector cells to the Fas receptor on the target cell surface may induce target cell apoptosis via activation of the Fas receptor signal transduction pathway. In one embodiment, the antibody of the invention induces FasL expression on effector cells. Various factors can affect ADCC, including the type of effector cells involved, cytokines (IL-2 and G-CSF, for example), incubation time, the number of receptors present on the surface of the cells, and antibody affinity.

In yet another embodiment, a method of inducing cell death by administering to a patient in need thereof an antibody of the present invention coupled or complexed to an agent, wherein the antibody-agent couple or complex enters the cell by internalization and the antibody-agent conjugate or complex is cleaved, releasing the agent is provided. Internalization can take place by any suitable means, for example, by endocytosis or by phagocytosis. The invention thus provides a means of treating a disease (e.g., treating can include ameliorating the effects of a disease, preventing a disease, or inhibiting the progress of a disease) in a patient.

Specifically, an antibody is used to introduce an agent into a cell. The antibody binds to proteins preferentially expressed on the surface of diseased cells, such as proteins with sulfated tyrosine residues. In a preferred embodiment, the agent is a toxin such as doxorubicin, morpholino-doxorubicin, or morpholino-daunorubicin. In a more preferred embodiment, the toxin is linked to the antibody via an adipic acid linker or an [N-ε-Maleimidocaproic acid hydrazide linker. The adipic acid linker has been used to bind to the a amino groups, whereas the N-[maleimidocaproic acid]hydrazide linker has been used to bind to both the a amino groups and also to the SH groups of the reduced disulphide linkages (via the maleimido group to form a C—S bond). In addition, a hydrazone bond is formed between the drug and the N-[maleimidocaproic acid]hydrazide linker.

After the antibody-agent complex binds to the cell surface protein, the cell internalizes the complex. Enzymes within the cell then cleave the antibody-toxin linkage, and the toxin acts on the cell to bring about its death. In another embodiment, the invention provides a composition for treating a disease comprising such an antibody-toxin conjugate.

Another embodiment provides an analogous method for introducing a non-toxic agent into a cell. The non-toxic agent can be used to change the behavior or activity of the cell, for example by directly or indirectly activating or repressing the activity of a specific gene.

In one other embodiment, the present invention provides a method of preventing infection by a virus comprising administering to a patient in need thereof an antibody as herein. Thus, a means of treating a disease is accomplished by administering an antibody that blocks infection. The cell expresses on its surface a protein containing a sulfated-tyrosine motif-containing epitope that is recognized by the antibody and that is also necessary for infection by the infectious agent. The antibody binds to the protein, thereby blocking infection. Proteins that the preferred antibody is known to bind via a sulfated tyrosine motif-containing epitope include fibrinogen γ chain, GPIb-α chain, complement C4, and PSGL-1. Proteins that the preferred antibody is believed to bind via a sulfated tyrosine motif—containing epitope include CCR5 and CXCR4. Either of CCR5 and CXCR4 can function as a coreceptor necessary for HIV infection. In a preferred embodiment, the antibody could be used to block infection by an HIV strain. Preferably the antibody is Y1.

Finally, a method is provided for introducing an agent into a cell that expresses sulfated PSGL-1 having the following steps: coupling or complexing the agent to an antibody as described herein and administering the antibody-agent couple or complex to the cell.

The antibody of the present invention binds to sulfated PSGL-1. White cells involved in inflammation, such as monocytes, neutrophils, and lymphocytes, are primarily recruited by the four adhesion molecules, PSGL-1, P-selectin, VLA-4, and VCAM-1 in the inflammatory processes of diseases such as atherosclerosis (Huo and Ley, Acta Physiol. Scand., 173: 35-43 (2001); Libby, Sci. Am. May: 48-55 (2002); Wang et al., J Am. Coll. Cardiol. 38: 577-582 (2001)). The antibody's interference with any of these central molecules may suggest a potential role for the antibody in abrogating related diseases. Specifically, P-selectin controls cell attachment and rolling. Additionally, P-selectin—PSGL-1 interactions activate a number of other molecules on cells which are integrally connected with tumorigenesis (when concerned with malignant cells) and inflammatory responses (when concerned with white blood cells) (Shebuski and Kilgore, J. Pharmacol. Exp. Ther. 300: 729-735 (2002)). Based on this understanding of P-selectin's ability to regulate cellular processes, it is apparent that the antibody's enhanced scFv selectivity for sulfated PSGL-1 may make it a superior molecule for treating a variety of malignant and inflammatory diseases. Moreover, models of malignant disease have shown that P-selectin binding to malignant cells requires sulfation of PSGL-1 (Ma and Geng, J. Immunol. 168: 1690-1696 (2002)). This requirement is similar to that for binding of the antibody. Thus, one can expect that the antibody could abrogate P-selectin facilitation of progressing malignant disease.

Preferably, the antibody of the present invention binds to an epitope present on at least one cell type involved in inflammation or tumorigenesis, including T-ALL cells, AML cells, Pre-B-ALL cells, B-leukemia cells, B-CLL cells, multiple myeloma cells, and metastatic cells. Further preferably, the antibody of the present invention may bind to epitopes on a lipid, carbohydrate, peptide, glycolipid, glycoprotein, lipoprotein, and/or lipopolysaccharide molecule. Such epitopes preferably have at least one sulfated moiety. Alternatively, but also preferably, the antibody of the present invention crossreacts with two or more epitopes, each epitope having one or more sulfated tyrosine residues, and at least one cluster of two or more acidic amino acids, an example of which is PSGL-1.

These antibodies, antigen-binding fragment or complex thereof, of the present invention may be internalized into a cell following binding to PSGL-1 on the surface of the cell. Such internalization may occur via endocytosis as an active process, which is manner, time and temperature dependent. For example, Y1 is specifically internalized into cells from AML patients via PSGL-1.

The antibody of the present invention binds to proteins having tyrosine sulfation sites. Such proteins include PSGL-1, GPIb, α-2antiplasmin; aminopeptidase B; CC chemokine receptors such as CCR2, CCR5, CCR3, CXCR3, CXCR4, CCR8, CCR2b, and CXCI; seven-transmembrane-segment (7TMS) receptors; coagulation factors such as factor V, VIII, and IX; fibrinogen gamma chain; heparin cofactor II; secretogranins such as secretogranin I and II; vitronectin, amyloid precursor, α-2-antiplasmin; cholecystokinin; α-choriogonadotropin; complement C4; dermatan sufaieproteiglycan; fibrinectin; and castrin. In a preferred embodiment, the antibody of the present invention binds to sulfated CC chemokine receptors such as CCR5, CXCR4, CXCI, and CCR2b. As mentioned previously, sulfated tyrosines may contribute to the binding of CCR5 to MIP-1α, MIPβ, and HIV-1 gp120/CD4 and to the ability of HIV-1 to enter cells expressing CCR5 and CD4.

Antibodies, peptides, polypeptides, proteins, and fragments and constructs thereof can be produced in either prokaryotic or eukaryotic expression systems. Methods for producing antibodies and fragments in prokaryotic and eukaryotic systems are well-known in the art.

A eukaryotic cell system, as defined in the present invention and as discussed, refers to an expression system for producing peptides or polypeptides by genetic engineering methods, wherein the host cell is a eukaryote. A eukaryotic expression system may be a mammalian system, and the peptide or polypeptide produced in the mammalian expression system, after purification, is preferably substantially free of mammalian contaminants. Other examples of a useful eukaryotic expression system include yeast expression systems.

A preferred prokaryotic system for production of the peptide or polypeptide of the invention uses E. coli as the host for the expression vector. The peptide or polypeptide produced in the E. coli system, after purification, is substantially free of E. coli contaminating proteins. Use of a prokaryotic expression system may result in the addition of a methionine residue to the N-terminus of some or all of the sequences provided for in the present invention. Removal of the N-terminal methionine residue, after peptide or polypeptide production to allow for full expression of the peptide or polypeptide, can be performed as is known in the art, one example being with the use of Aeromonas aminopeptidase under suitable conditions (U.S. Pat. No. 5,763,215).

The antibodies and polypeptides of the subject invention can be complexed with e.g. associated with, combined, fused, or linked to various pharmaceutical agents, such as drugs, toxins, and radioactive isotopes and optionally, with a pharmaceutically effective carrier, to form peptide-drug compositions comprising an antibody/polypeptide and a pharmaceutical agent having anti-disease and/or anti-cancer activity. Such compositions may also be used for diagnostic purposes.

For example, conjugation or complexing of anthracyclines to antibodies is generally known in the art (Dubowchik & Walker, Pharmacol. & Thera. 83: 67-123 (1999); Trail et al., Cancer Immunol. Immunother. 52: 328-337 (2003)). Such conjugation can be by direct conjugation or via linkers, such as acid cleavable linkers or enzyme cleavable linkers and may involve the use of intermediate carriers such as dextran and synthetic polymers. Anthracyclines have been complexed to the antibodies of the present invention via (1) α amino groups (about pH 8) to produce a drug:antibody ratio of 4:1 (in which case two drug molecules are attached to the heavy chain and two to the light chain); and (2) disulfide linkages to produce a drug antibody ratio of between 4:1 and 8:1 depending or the method used. As is known in the art, the drug antibody ratio can, for example, be doubled, tripled or quadrupled, etc, by using a two, three, four, etc., branched linker. One skilled in the art may make chemical modifications to the antibody, linker, carrier and/or drug in order to make reactions more convenient for the purposes of preparing a conjugate.

In one embodiment of the present invention, the two disulfide linkages in the Fc region were reduced with mercaptoethylamine and was then reacted with the drug linker at about pH 7, which leads to a drug antibody ratio of 4:1 (in which case all the four drugs are attached in the heavy chains). In another embodiment, the four disulfide bonds in the hinge region were reduced with DTT (about pH 7) and was then reacted with the drug linker, which leads to a drug antibody ratio of about 7:1 to 8:1 (in which case 5 or 6 of the drug molecules are attached to the heavy chains and one or two of the drug molecules are attached to the light chains).

Examples of carriers useful in the invention include dextran, HPMA (a hydrophilic polymer), or any other polymer, such as a hydrophilic polymer, as well as derivatives, combinations and modifications thereof. Alternatively, decorated liposomes, also known as immunoliposomes, can be used, such as liposomes decorated with scFv Y1 molecules, such as Doxil, a commercially available liposome containing large amounts of doxorubicin. Such liposomes can be prepared to contain one or more desired agents and be admixed with the antibodies of the present invention to provide a high drug to antibody ratio.

Alternatively, the link between the antibody or polypeptide and the agent may be a direct link. A direct link between two or more neighboring molecules may be produced via a chemical bond between elements or groups of elements in the molecules. The chemical bond can be, for example, an ionic bond, a covalent bond, a hydrophobic bond, a hydrophilic bond, an electrostatic bond, or a hydrogen bond. The bonds can be, for example, amide, carbon-sulfide, peptide, and/or disulfide bonds. In order to attach the the antibody to the agent or linker, amine, carboxy, hydroxyl, thiol and ester functional groups may be used, as is known in the art to form covalent bonds.

The link between the peptide and the agent or between the peptide and carrier, or between the carrier and agent may be via a linker compound. As used herein, a linker compound is defined as a compound that joins two or more moieties. The linker can be straight-chained or branched. A branched linker compound may be composed of a double-branch, triple branch, or quadruple or more branched compound. Linker compounds useful in the present invention include those selected from the group having dicarboxylic acids, malemido hydrazides, PDPH, carboxylic acid hydrazides, and small peptides.

More specific examples of linker compounds useful, according to the present invention, include: (a) dicarboxylic acids such as succinic acid, glutaric acid, and adipic acid; (b) maleimido hydrazides such as N-[maleimidocaproic acid) hydrazide, 4-[N-maleimidomethyl]cyclohexan-1-carboxylhydrazide, and N-[maleimidoundecanoic acid]hydrazide; (c) (3-[2-pyridyldithio]propionyl hydrazide)derivatives, combinations, modifications and analogs thereof; and (d) carboxylic acid hydrazides selected from 2-5 carbon atoms.

Linking via direct coupling using small peptide linkers is also useful. For example, direct coupling between the free sugar of, for example, the anti-cancer drug doxorubicin and a scFv may be accomplished using small peptides. Examples of small peptides include AU1, AU5, BTag, c-myc, FLAG, Glu-Glu, HA, His6, HSV, HTTPHH, IRS, KT3, Protein C, S-TAG®, T7, V5, VSV-G, and KAK.

Antibodies and polypeptides of the present invention may be bound to, conjugated to, complexed with, or otherwise associated with imaging agents (also called indicative markers), such as radioisotopes, and these conjugates can be used for diagnostic and imaging purposes. Kits having such radioisotope-antibody (or fragment) complexes are provided.

Examples of radioisotopes useful for diagnostics include ¹¹¹indium, ¹¹³indium, ^{99m}rhenium, ¹⁰⁵rhenium, ¹⁰¹rhenium, ^{99m}technetium, ^{121m}tellurium, ^{122m}tellurium, ^{125m}telluriunm ¹⁶⁵thulium, ¹⁶⁷thulium ¹⁶⁸thulium ¹²³iodine, ¹²⁶iodine, ¹³¹iodine, ¹³³iodine, ^{81m}kypton, ³³xenon, ⁹⁰yttrium, ²¹³bismuth, ⁷⁷bromine, ¹⁸fluorine, ⁹⁵ruthenium, ⁹⁷ruthenium, ¹⁰³rutheum, ¹⁰⁵uthenium, 107mercury, ²⁰³mercury, ⁶⁷gallium, and ⁶⁸gallium. Preferred radioactive isotopes, are opaque to X-rays or any suitable paramagnetic ions.

The indicative marker molecule may also be a fluorescent marker molecule. Examples of fluorescent marker molecules include fluorescein, phycoerythrin, or rhodamine, or modifications or conjugates thereof.

Antibodies and polypeptides conjugated to indicative markers may be used to diagnose, prognose, or monitor disease states. Generally, such methods include providing a sample of at least one cell from a patient and determining whether the antibody or fragment thereof of the present invention binds to the cell of the patient, thereby indicating that the patient is at risk for or has the disease. Such monitoring may be carried out in vivo, in vitro, or ex vivo. Where the monitoring or diagnosis is carried out in vivo or ex vivo, the imaging agent is preferably physiologically acceptable in that it does not harm the patient to an unacceptable level. Acceptable levels of harm may be determined by clinicians using such criteria as the severity of the disease and the availability of other options.

With respect to cancer, staging a disease in a patient generally involves determining the classification of the disease based on the size, type, location, and invasiveness of the tumor. One classification system to classify cancer by tumor characteristics is the “TNM Classification of Malignant Tumours” (6th Edition) (L. H. Sobin, Ed.), which is incorporated by reference herein and which classifies stages of cancer into T, N, and M categories with T describing the primary tumor according to its size and location, N describing the regional lymph nodes, and M describing distant metastases. In addition, the numbers I, II, III and IV are used to denote the stages and each number refers to a possible combination of TNM factors. For example, a Stage I breast cancer is defined by the TMN group: T1, N0, M0 which mean:T1—Tumor is 2 cm or less in diameter, N0—No regional lymph node metastasis, M0—No distant metastasis. Another system is used to stage AML, with subtypes of classified based on the French-American-British system using the morphology observed under routine processing and cytochemical staining.

In addition, a recently proposed World Health Organization (WHO) staging or classification of neoplastic diseases of the hematopoietic and lymphoid tissues includes (specifically for AMLs) traditional FAB-type categories of disease, as well as additional disease types that correlate with specific cytogenetic findings and AML associated with myelodysplasia. Others have also proposed pathologic classifications. For example, one proposal specific for AML includes disease types that correlate with specific cytogenetic translocations and can be recognized reliably by morphologic evaluation and immunophenotyping and that incorporate the importance of associated myelodysplastic changes. This system would be supported by cytogenetic or molecular genetic studies and could be expanded as new recognizable clinicopathologic entities are described (Arber, Am. J. Clin. Pathol. 115(4): 552-60 (2001)).

The present invention provides for a diagnostic kit for in vitro analysis of treatment efficacy before, during, or after treatment, having an imaging agent having a peptide of the invention linked to an indicative marker molecule, or imaging agent. The invention further provides for a method of using the imaging agent for diagnostic localization and imaging of a cancer, more specifically a tumor, having the following steps: (a) contacting the cells with the composition; (b) measuring the radioactivity bound to the cells; and hence (c) visualizing the tumor.

Examples of suitable imaging agents include fluorescent dyes, such as FITC, PE, and the like, and fluorescent proteins, such as green fluorescent proteins. Other examples include radioactive molecules and enzymes that react with a substrate to produce a recognizable change, such as a color change.

In one example, the imaging agent of the kit is a fluorescent dye, such as FITC, and the kit provides for analysis of treatment efficacy of cancers, more specifically blood-related cancers, e.g., leukemia, lymphoma, and myeloma. FACS analysis is used to determine the percentage of cells stained by the imaging agent and the intensity of staining at each stage of the disease, e.g., upon diagnosis, during treatment, during remission and during relapse.

Antibodies and polypeptides of the present invention may be bound to, conjugated to, or otherwise associated with anti-cancer agents, anti-neoplastic agents, anti-viral agents, anti-metastatic agents, anti-inflammatory agents, anti-thrombosis agents, anti-restenosis agents, anti-aggregation agents, anti-autoimmune agents, anti-adhesion agents, anti-cardiovascular disease agents, pharmaceutical agents, or other anti-disease. An agent refers to an agent that is useful in the prophylactic treatment or diagnosis of a mammal including, but not restricted to, a human, bovine, equine, porcine, murine, canine, feline, or any other warm-blooded animal.

Examples of such agents include, but are not limited to, anti-viral agents including acyclovir, ganciclovir and zidovudine; anti-thrombosis/restenosis agents including cilostazol, dalteparin sodium, reviparin sodium, and aspirin; anti-inflammatory agents including zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid; anti-autoimmune agents including leflunomide, denileukin diftitox, subreum, WinRho SDF, defibrotide, and cyclophosphamide; and anti-adhesion/anti-aggregation agents including limaprost, clorcromene, and hyaluronic acid.

Exemplary pharmaceutical agents include anthracyclines such as doxorubicin (adriamycin), daunorubicin, idarubicin, detorubicin, carminomycin, epirubicin, esorubicin, morpholinodoxorubicin, morpholinodaunorubicin, methoxymorpholinyldoxorubicin, methoxymorpholinodaunorubicin and methoxymorpholinyldoxorubicin and substituted derivatives, combinations and modifications thereof. Further exemplary pharmaceutical agents include cis-platinum, taxol, calicheamicin, vincristine, cytarabine (Ara-C), cyclophosphamide, prednisone, fludarabine, idarubicin, chlorambucil, interferon alpha, hydroxyurea, temozolomide, thalidomide and bleomycin, and derivatives, combinations and modifications thereof.

An anti-cancer agent is an agent with anti-cancer activity. For example, anti-cancer agents include agents that inhibit or halt the growth of cancerous or immature pre-cancerous cells, agents that kill cancerous or pre-cancerous cells, agents that increase the susceptibility of cancerous or pre-cancerous cells to other anti-cancer agents, and agents that inhibit metastasis of cancerous cells. In the present invention, an anti-cancer agent may also be an agent with anti-angiogenic activity that prevents, inhibits, retards, or halts vascularization of tumors.

Inhibition of growth of a cancer cell includes, for example, the (i) prevention of cancerous or metastatic growth, (ii) slowing down of the cancerous or metastatic growth, (iii) the total prevention of the growth process of the cancer cell or the metastatic process, while leaving the cell intact and alive, (iv) interfering contact of cancer cells with the microenvironment, or (v) killing the cancer cell. For example, an antibody could effect the killing of a cancer cell by binding to the cancer cell and thereby stimulating T cells or natural killer cells to kill the bound cell by antibody-dependent cell cytotoxicity.

An anti-leukemia agent is an agent with anti-leukemia activity. For example, anti-leukemia agents include agents that inhibit or halt the growth of leukemic or immature pre-leukemic cells, agents that kill leukemic or pre-leukemic, agents that increase the susceptibility of leukemic or pre-leukemic cells to other anti-leukemia agents, and agents that inhibit metastasis of leukemic cells. In the present invention, an anti-leukemia agent may also be agent with anti-angiogenic activity that prevents, inhibits, retards or halts vascularization of tumors.

Inhibition of growth of a leukemia cell includes, for example, the (i) prevention of leukemic or metastatic growth, (ii) slowing down of the leukemic or metastatic growth, (iii) the total prevention of the growth process of the leukemia cell or the metastatic process, while leaving the cell intact and alive, (iv) interfering contact of cancer cells with the microenvironment, or (v) killing the leukemia cell.

Examples of anti-disease, anti-cancer, and anti-leukemic agents to which antibodies and fragments of the present invention may usefully be linked include toxins, radioisotopes, and pharmaceuticals.

Examples of toxins include gelonin, Pseudomonas exotoxin (PE), PE40, PE38, diphtheria toxin, ricin, or derivatives, combinations and modifications thereof.

Examples of radioisotopes include gamma-emitters, positron-emitters, and x-ray emitters that may be used for localization and/or therapy, and beta-emitters and alpha-emitters that may be used for therapy. The radioisotopes described previously as useful for diagnostics are also useful for therapeutics.

Non-limiting examples of anti-cancer or anti-leukemia agents include anthracyclines such as doxorubicin (adriamycin), daunorubicin, idarubicin, detorubicin, carminomycin, epirubicin, esorubicin, morpholinodoxorubicin, morpholinodaunorubicin,methoxymorpholinyldoxorubicin,methoxymorpholinodaunorubic in and methoxymorpholinyldoxorubicin and substituted derivatives, combinations and modifications thereof. Exemplary pharmaceutical agents include cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide, and bleomycin, and derivatives, combinations and modifications thereof.

In one embodiment, the pharmaceutical compositions of the present invention have an antibody or polypeptide of the present invention and a pharmaceutically acceptable carrier. The antibody or polypeptide can be present in an amount effective to inhibit cell rolling, inflammation, auto-immune disease, metastasis, growth and/or replication of tumor cells or leukemia cells, or increase in number of tumor cells in a patient having a tumor or leukemia cells in a patient having leukemia. Alternatively, the antibody or polypeptide can be present in an amount effective to increase mortality of tumor cells or leukemia cells. Also alternatively, the antibody or polypeptide can be present in an amount effective to alter the susceptibility of diseased cells to damage by anti-disease agents, tumor cells to damage by anti-cancer agents, or leukemia cells to damage by anti-leukemia agents. Further alternatively, the antibody or polypeptide can be present in an amount effective to decrease number of tumor cells in a patient having a tumor or leukemia cells in a patient having leukemia. Yet further alternatively, the antibody or polypeptide can be present in an amount effective to inhibit restenosis. The antibody, or polypeptide can also be present in an amount effective to inhibit HIV entry. Alternatively, the antibody or polypeptide, can be used as a targeting agent to direct a therapeutic to a specific cell or site.

Antibodies and polypeptides of the present invention may be administered to patients in need thereof via any suitable method. Exemplary methods include intravenous, intramuscular, subcutaneous, topical, intratracheal, intrathecal, intraperitoneal, intralymphatic, nasal, sublingual, oral, rectal, vaginal, respiratory, buccal, intradermal, transdermal, or intrapleural administration.

For intravenous administration, the formulation preferably will be prepared so that the amount administered to the patient will be an effective amount from about 0.1 mg to about 1000 mg of the desired composition. More preferably, the amount administered will be in the range of about I mg to about 500 mg of the desired composition. The compositions of the invention are effective over a wide dosage range and depend on factors such as the particulars of the disease to be treated, the half-life of the peptide, or polypeptide-based pharmaceutical composition in the body of the patient, physical and chemical characteristics of any agent complexed to antibody or fragment thereof and of the pharmaceutical composition, mode of administration of the pharmaceutical composition, particulars of the patient to be treated or diagnosed, as well as other parameters deemed important by the treating physician.

Pharmaceutical composition for oral administration may be in any suitable form. Examples include tablets, liquids, emulsions, suspensions, syrups, pills, caplets, and capsules. Methods of making pharmaceutical compositions are well known in the art (See, e.g., Remington, The Science and Practice of Pharmacy, Alfonso R. Gennaro (Ed.) Lippincott, Williams & Wilkins (pub)).

The pharmaceutical composition may also be formulated so as to facilitate timed, sustained, pulsed, or continuous release. The pharmaceutical composition may also be administered in a device, such as a timed, sustained, pulsed, or continuous release device.

The pharmaceutical composition for topical administration can be in any suitable form, such as creams, ointments, lotions, patches, solutions, suspensions, lyophilizates, and gels.

Compositions having antibodies, constructs, conjugates, and fragments of the subject invention may comprise conventional pharmaceutically acceptable diluents, excipients, carriers, and the like. Tablets, pills, caplets, and capsules may include conventional excipients such as lactose, starch, and magnesium stearate. Suppositories may include excipients such as waxes and glycerol. Injectable solutions comprise sterile pyrogen-free media such as saline, and may include buffering agents, stabilizing agents or preservatives. Conventional enteric coatings may also be used.

The antibodies and polypeptides of the present invention and pharmaceutical compositions thereof, can be used in methods of ameliorating the effects of a disease, preventing a disease, treating a disease, or inhibiting the progress of a disease in patients in need thereof. Such methods include inhibiting cell rolling, inflammation, autoimmune disease, metastasis, growth and/or replication of tumor cells or leukemia cells, or increase in number of tumor cells in a patient having a tumor or leukemia cells in a patient having leukemia. In addition, such methods include increasing the mortality rate of tumor cells or leukemia cells, alter the susceptibility of diseased cells to damage by anti-disease agents, tumor cells to damage by anti-cancer agents, or leukemia cells to damage by anti-cancer agents. Such methods also include decreasing number of tumor cells in a patient having tumor or leukemia cells in a patient having leukemia. Such methods also include inhibiting or decreasing HIV entry in cells. Such methods further include preventing or inhibiting cardiovascular diseases such as restenosis.

The present invention moreover provides a method of manufacturing a medicament for the treatment of various disease states such as, e.g., AML, T-ALL, B-leukemia, B-CLL, Pre-B-ALL, multiple myeloma, metastasis, HIV infection, cardiovascular diseases, or other diseases in which such cellular functions or actions as cell rolling, inflammation, immune reactions, infection, autoimmune reactions, metastasis, play a significant role. Such medicament comprises the antibodies and the polypeptides of the present invention.

In one embodiment, the invention provides a method of diagnosing cancer in a person by assaying the ability of Y1 to bind specifically to a tissue sample and comparing Y1 binding to binding by a control antibody such as KPL-1. In one embodiment, the method comprises isolating cell samples from blood or solid tissue from the person, incubating the cells with an antibody or a fragment thereof that recognizes a sulfated tyrosine motif-containing epitope (“the experimental antibody”), washing away the non-specifically bound antibody, and comparing the results to those of a corresponding staining procedure performed with a reference standard such as a control antibody with known binding activity. A control antibody is one that recognizes an epitope containing the unsulfated form of the tyrosine motif or antigens that contain such. The presence of tumor cells is indicated when the experimental antibody binding is substantially greater than binding by the control, as determined by the strength of the staining. The staining procedure can be performed by standard methods. For example, the first antibody can be visualized by using secondary antibodies that recognize the first antibody and that are conjugated to an enzyme substrate which produces a color reaction when acted on by the enzyme. Alternatively, the presence of tumor cells is indicated when both the experimental antibody and the control antibody bind to the cells, but the cells internalize Y1 and do not internalize the control antibody. In one embodiment, the cancer is a solid tumor. In another embodiment, the cancer is a blood-borne tumor. In a preferred embodiment, the experimental antibody is Y1 or a fragment thereof, or a related antibody or a fragment thereof. In another preferred embodiment, the control antibody is KPL1.

In another embodiment, the invention provides a method of diagnosing a cancer comprising screening cell samples from blood or solid tissue for the presence of tumor cells. Western blots are performed on cell sample lysates using Y1 or a fragment thereof, or a related antibody or a fragment thereof. Y1 binding can be observed by tagging Y1 itself with a detectable label, or by using standard methods that employ a detectable anti-human antibody. The presence of tumor cells is indicated when Y1 binding is substantially greater than binding by the control, where the control is defined as above. The presence of tumor cells is indicated when Y1 binding is substantially greater than binding by the control.

In another embodiment, the invention provides a method of identifying protein markers of blood-borne or solid tumors by preparing a cell lysate and purifying the lysate by passing it through an affinity column. The affinity column incorporates Y1 or a fragment thereof, or a related antibody or fragment thereof. In one embodiment, the cell lysate is derived from a primary tissue sample collected from a human being. In another embodiment, the cell lysate is derived from a tumor cell line. In a further embodiment, the tumor cell line can be an immortalized cell line.

In another embodiment, the invention provides a method of monitoring the stage of a blood-borne cancer comprising isolating white blood cells from a patient with a blood-borne cancer, incubating the cells with Y1, determining the extent of Y1 binding relative to reference standard.

As an alternative approach for therapeutic targeting of sulfated tyrosine epitopes present on proteins, such as GPIb and PSGL-1, a small inorganic chemical entity may be identified by screening of an appropriate combinatorial library. Such a chemical entity may have a number of advantages over a scFv or IgG-based therapeutic agent. For example, an inorganic chemical entity may be administered orally and have an enhanced biosafety profile, including reduced immuno-crossreactivity. It may provide enhanced selectivity towards the target, particularly following rational drug design to optimize an initially selected lead compound. Other advantages include lower production costs, longer shelf-life and a less complicated regulatory approval process.

Since a number of embodiments of the epitope of the invention have been identified, e.g. on GPIb and PSGL-1, a ligand-driven approach may be taken to identify inorganic chemical entities, which have very narrow specificity, or alternately, target more than one sulfated tyrosine epitope for disease states such as re-perfusion injury which involves more than one distinct target each bearing such an epitope. The ligand-driven approach significantly shortens the screening process for identifying targets for therapeutic intervention, and enables simultaneous target validation with lead optimization, which may be carried out with a series of focused libraries.

A library of inorganic chemical entities specialized for targeting sulfated tyrosine epitopes may be designed and developed first by analyzing the three dimensional interaction between an antibody such as Y1 and its known targets such as residues sulfated Tyr-276 and Asp-277 of GPIb. Chemical libraries composed of entities that mimic the Y1 binding site and which provide increased affinity to the target may be developed by computer assisted combinatorial library design.

Throughout this application, reference has been made to various publications, patents, and patent applications. The teachings and disclosures of these publications, patents, and patent applications in their entireties are hereby incorporated by reference into this application to more fully describe the state of the art to which the present invention pertains.

### EXAMPLES

The following examples are set forth to aid in understanding the invention but are not intended and should not be construed, to limit its scope in any way. Although specific reagents and reaction conditions are described, modifications can be made that are meant to be encompassed by the scope of the invention. The following examples, therefore, are provided to further illustrate the invention.

### Example 1

### Identification of Y1 Ligand from Primary AML Cells (R1198-3)

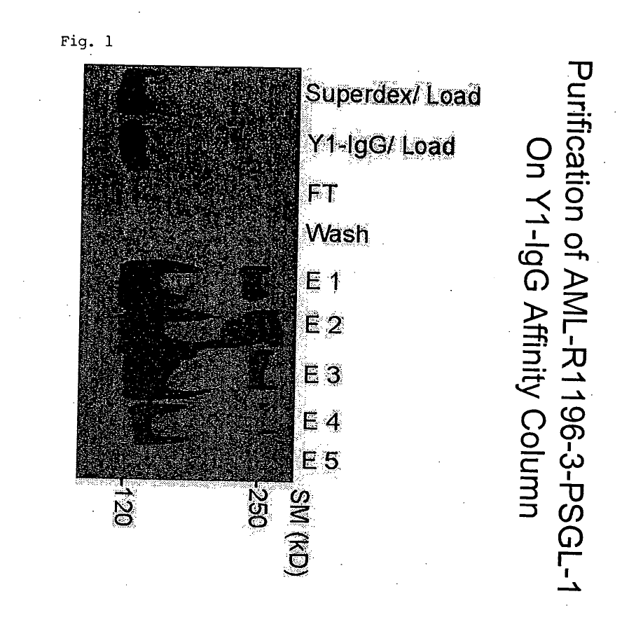

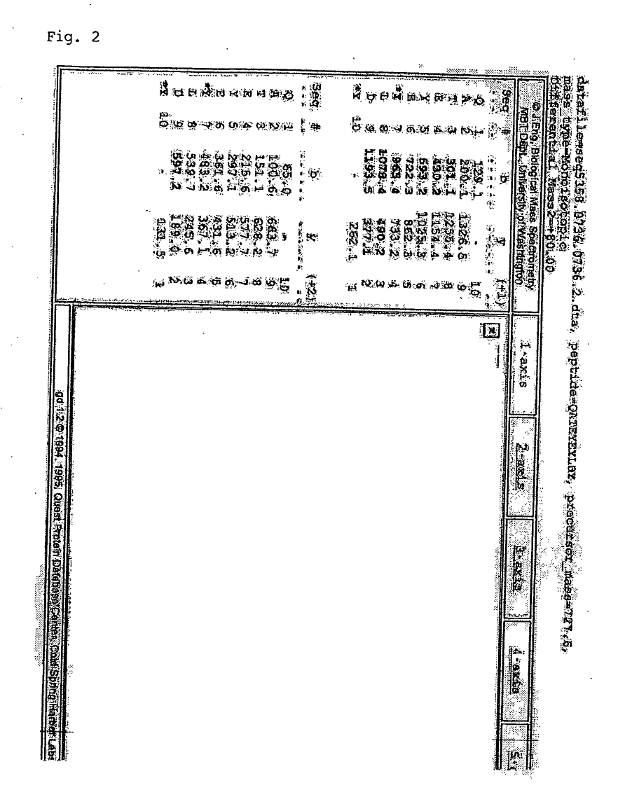

1.1 Primary AML cells (stage M4) were collected from a patient and lysed. The lysate was subjected to purification comprising affinity chromatography on a Y1-IgG column (see FIG. 1). The isolated protein was digested with endoproteinase Asp-N, and the resulting peptide sequence was determined using mass spectrometry. The sequence was identical to the published human PSGL-1 N-terminal amino acid sequence. These results indicate that primary AML cells at stage 4 express PSGL-1 that can be bound by Y1-IgG. It was further determined that the purified protein was sulfated at tyrosines 2 and 3 of the Y1 recognition motif (see FIG. 2). Internal controls were used to verify the specificity of the immunomodulatory effects of Y1 e.g. no induction of mouse interleukin-6 secretion was detected.

### Example 2

### Antibody-Dependent Cell Cytotoxicity

2.1 Effect of Y1-IgG:

Studies to determine whether Y1-IgG is capable of mediating antibody dependent cell cytotoxicity (ADCC) have shown this antibody mediates effector cell cytotoxicity of various target cells, including ML2 (an AML-derived cell line which served as a target in our model system) and B-CLL cells from patient clinical samples. Y1-IgG binds these cell types via CD162 (PSGL-1), a molecule which is substantially absent on healthy B-cells and early stage AML.

The effector cell populations that are involved in Y1-IgG ADCC have been defined. For Y1-IgG to mediate ADCC, natural killer (NK) cells (CD56+), γδT cells, and monocytes (CD14+) are required, but T-helper cells (CD4+) and cytotoxic T cells (CD8+) are not required. This was confirmed with donor cells from both healthy subjects and B-CLL patients.

Furthermore, even in the absence of target cells, Y1-IgG mediates activation of different types of effector cells, as measured by the appearance of an early activation marker (CD69+), secretion of cytokines, such as TNFα and IFNγ and induction of FasL. Hyper cross-linking (XL) of Y1-IgG with secondary anti-human Fc antibodies demonstrated that an apoptotic mechanism also contributes to cell killing.

Y1-IgG activity towards primary B-CLL cells in vitro was compared to that of two commercially available humanized antibodies currently used extensively for treatment of various lymphoid malignancies: Rituximab (which binds CD20) and Campath (which binds CD52). While the mechanism of action of Rituximab against B-CLL is not clear, its cytotoxic effects against CD20-positive malignant B cells may involve one or more of complement-dependent cytotoxicity (CDC), ADCC and induction of apoptosis. The cytotoxic effects of Campath against CD52-positive malignant B cells, as well as normal B and T cells, involves CDC, ADCC and induction of apoptosis. Campath administration is associated with complete ablation of all mature normal B and T cells, leading to severe hematological toxicity.

For ADCC experiments, mononuclear effector and target cells were separated on FICOLL®. Target cells were then labeled with PKH26, which stably incorporates a fluorescent dye within the lipid regions of the cell membranes. Cells were then washed and incubated with effector cells at various Effector:Target (E:T) ratios, in the absence or presence of different concentrations of Y1-IgG or control antibodies for 24 hours. Dead cells were stained by TOPRO®(Molecular Probes, Inc., Eugene, Oreg.): and analyzed by FACS on gated target cells.

For CDC experiments, mononuclear cells from B-CLL patients were separated on FICOLL®. Cells were incubated with or without Y1-IgG or control antibodies for 24 hours in the presence or absence of the patient's plasma. Apoptotic cells were then stained with Annexin-PI and analyzed by FACS.

To assess effector cell activation, mononuclear cells from healthy donors were separated on FICOLL®. Cells were incubated with or without Y1-IgG or control antibodies for 24 hours. FACS analysis with aCD69 (an early activation marker) antibody was performed for different types of effector cells. Secretion of cytokines such as TNFα and IFNγ were measured by ELISA.

For apoptosis experiments, mononuclear cells from B-CLL patients were separated on FICOLL®. Cells were incubated in the presence or absence of Y1-IgG or control antibodies for 10 minutes at 37° C. Anti-human Fc antibodies were then added and incubated for 4-24 hours at 37° C. Diseased cells (CD19+, CD5+) were then stained for apoptotic markers, Annexin-TOPRO® and analyzed by FACS.

Comparative studies with Y1-IgG and Rituximab indicate that Y1-IgG is superior to Rituximab with respect to mediation of ADCC and induction of apoptosis against B-CLL cells. In contrast to Campath®, Y1-IgG was found to be incapable of mediating CDC against primary B-CLL cells. These in vitro results indicate that Y1-IgG may be useful as a therapeutic agent for treatment of B-CLL based on its ADCC activity.

2.1.1 ADCC in Primary B-Chronic Lymphocytic Leukemia (B-CLL):

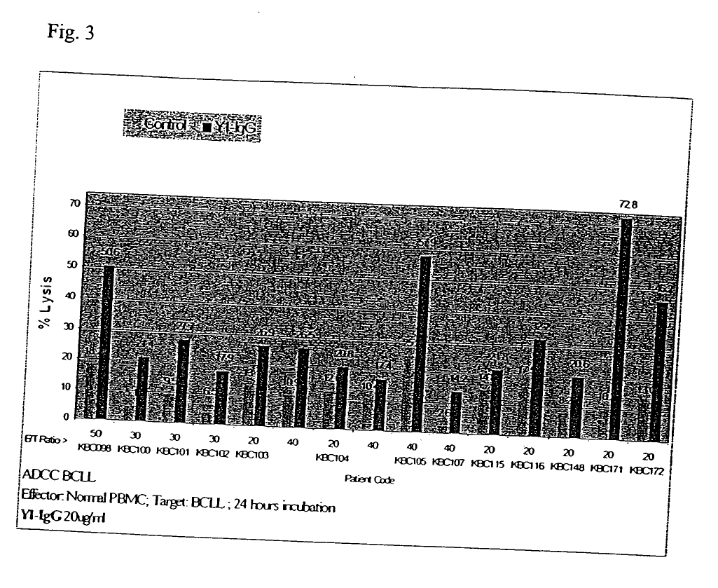

To determine if Y1-IgG mediates ADCC in primary B-CLL, B-CLL cells from different patients were co-incubated for 24 hours with PBMC effector cells at different effector/target cell ratios. Analysis of thirteen different B-CLL clinical samples indicated that Y1-IgG mediated effector cell cytotoxicity in all cases (FIG. 3) with the average extent of cell lysis about 21.4%. Four of thirteen samples (30%) exhibited more than 30% lysis, while only two of thirteen samples (15%) exhibited less than 10% lysis. In some cases, a high degree of lysis was seen even at a low E:T ratio, e.g. KBC171, which exhibited about 62% lysis, while in other cases, a low degree of lysis was seen even at a high E:T ratio, e.g. KBC104, which exhibited only about 7% lysis. Variation may be attributed to the effector cell samples obtained from different healthy donors, as well as to differences among the B-CLL samples.

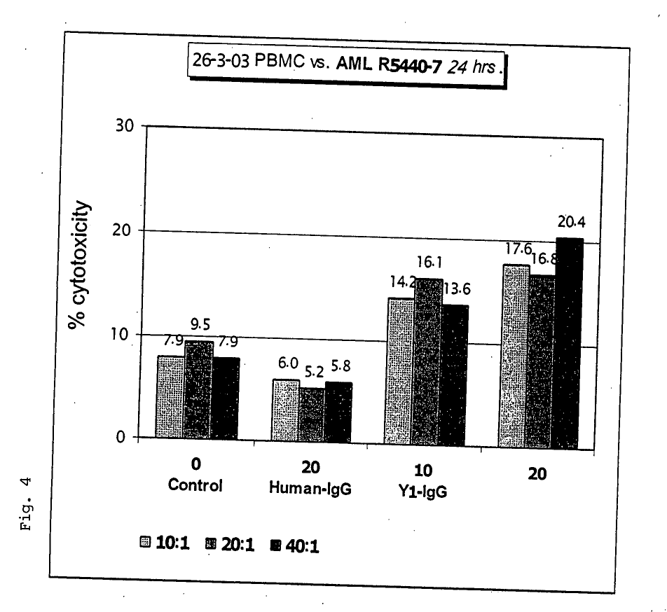

2.1.2 Y1-IgG-mediated ADCC by PBMC against acute myeloid leukemia cells: PBMC also effected ADCC against primary AML cells from a patient using varying ratios of PBMC:AML (10, 20, or 40: in the presence of 10 or 20 μg/ml Y1-IgG. For example, at a cell ratio of 10:1, 7.9% of AML cells died in the absence of antibody and 6% died in the presence of human IgG. In the presence of 10 and 20 μg/ml Y1-IgG 14.2% and 17.6% of the AML cells died, respectively (FIG. 4). The degree of cytotoxicity increased with increasing Y1-IgG concentration (10 or 20 μg/ml). Human IgG did not induce ADCC. A similar result was obtained for one additional primary AML sample (data not shown).

ML-2 cells provide a good model for ADCC since Y1-IgG binds without undergoing detectable internalization.

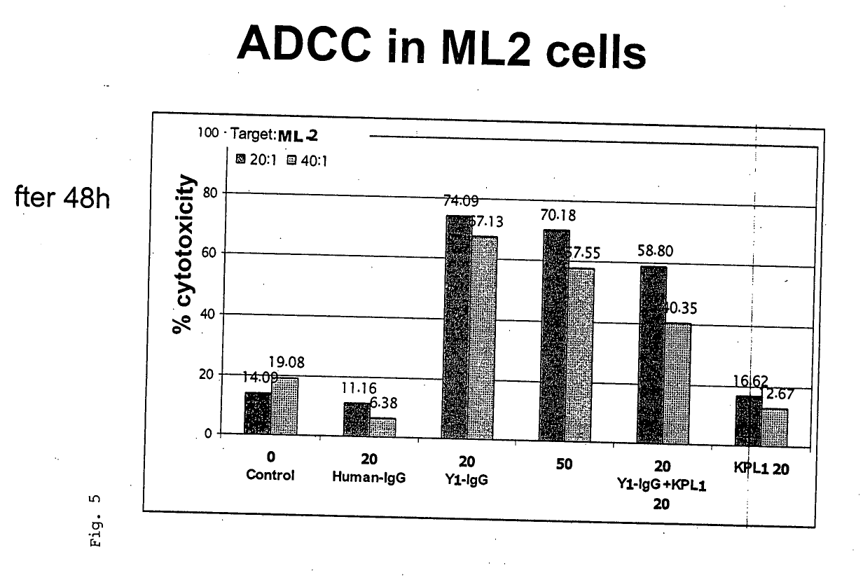

a. ML2 ADCC increases with Y1-IgG concentration: After 24 hours of incubation, cytotoxicity was higher in the presence of Y1-IgG than in its absence at four different effector (PBMC) to target ratios (5:1, 10:1, 20:1, 40:1) (FIG. 5). This effect was diminished or absent when mouse anti-PSGL-1 antibody KPL1 was substituted for Y1-IgG and diminished even further when human IgG (which binds to the Fc receptor on effector cells) was used instead of Y1-IgG. A Y1-IgG concentration as low as 5 μg/ml could induce ADCC when the effector:target ratio was 40:1.

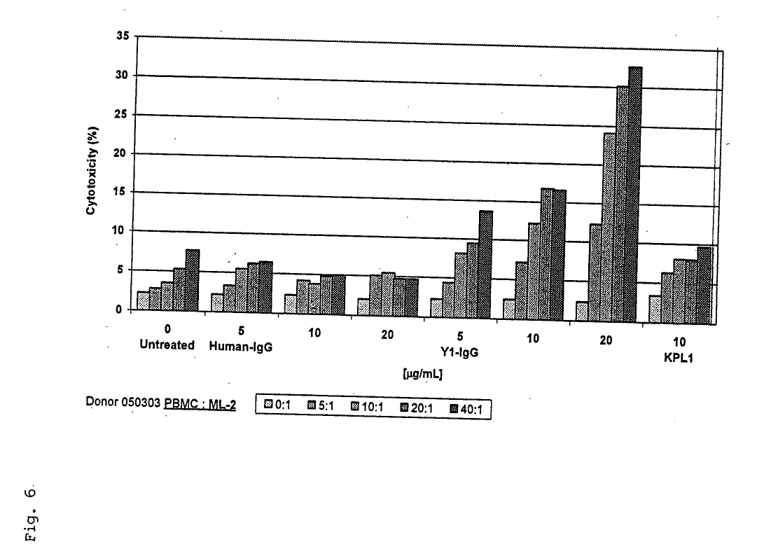

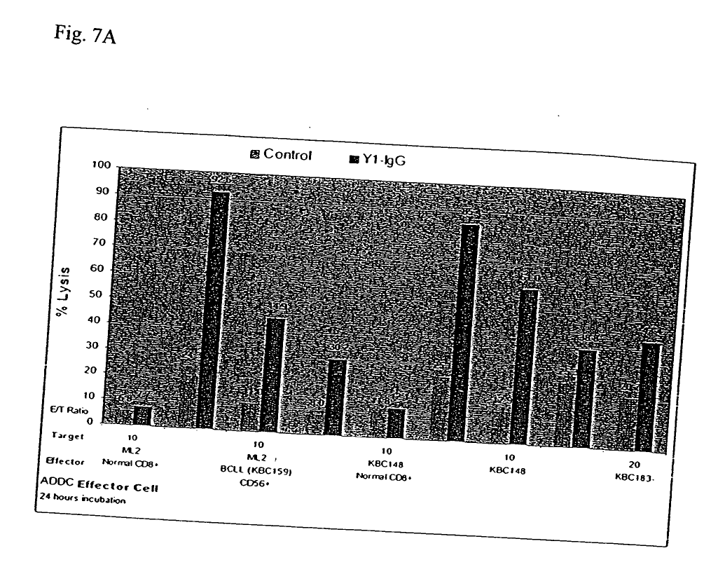

b. Competition between Y1-IgG and KPL-1: Y1-IgG (20 and 50 μg/ml) induced ADCC against ML2 cells at effector:target ratios of 20:1 and 40:1. The mouse anti-PSGL-1 antibody KPL-1 alone did not induce ADCC, and could therefore be used as a competitor for Y1-binding induced ADCC. KPL-1 partially inhibited Y1-IgG-induced ADCC (FIG. 6), and for example, while 74.1% cytotoxicity was observed after 48 hours incubation in the presence of Y1-IgG (20 μg/ml; effector/target ratio 20:1), the additional presence of KPL1 (20 μg/ml) resulted in only 58.8% cytotoxicity. Thus, Y1-IgG-induced ADCC of ML2 involves binding of PSGL-1 by Y1-IgG.

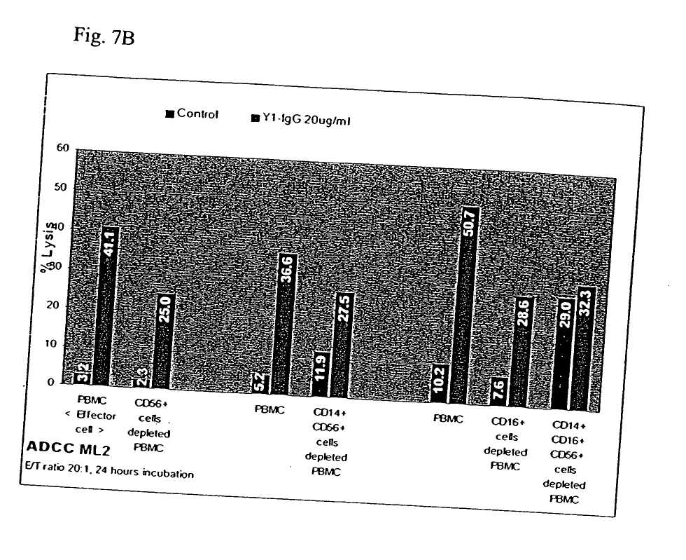

c. Involvement of Natural Killer, γδT cells and Monocytes in Y1-IgG Mediated ADCC: Positively selected effector cells were analyzed for their capability of effecting Y1-IgG mediated ADCC of ML2 or B-CLL target cells. Natural killer (NK) cells (CD56+), γδT cells and cytotoxic T-cells (CD8+) from normal donors and B-CLL patients were isolated using commercially available magnetic beads. As shown in FIG. 7A, NK cells from both normal donors and from B-CLL patients (KCS samples in FIG. 7A) are capable of effecting ADCC on ML2 and B-CLL targets (KCS samples in FIG. 7A), resulting in 13 to 68% lysis over control. Also, γδT cells were shown to mediate ADCC of ML2 cells. In contrast, cytotoxic T-cells do not appear to be involved in Y1-IgG mediated cytotoxicity.

Negative selection of specific cell populations from PBMC showed that CD14+ cells (monocytes) are also involved in ADCC against ML2 targets, in addition to NK cells (CD56+) and γδ+T cells (FIG. 7B). All of the effector cells which are involved in Y1-IgG mediated toxicity express the Fc receptor, CD16.

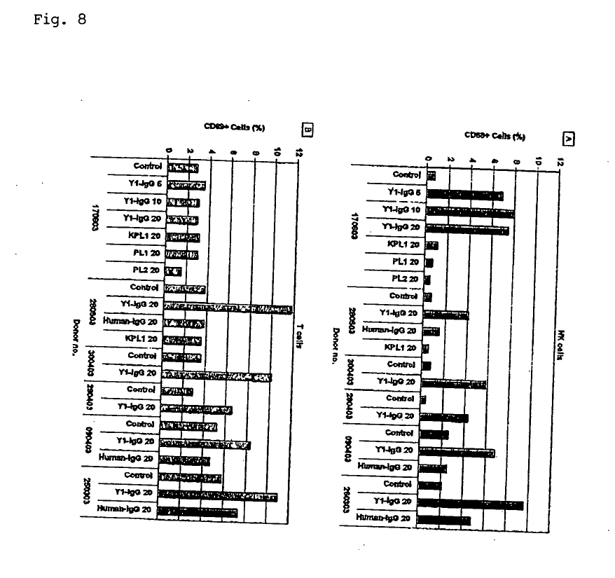

d. Activation of NK -cells by Y1: Y1 mediates ADCC by natural killer cells, as measured by expression of CD69. Effector cells from six healthy donors were incubated for 24 hours at 37° C. in the presence of Y1-IgG or human IgG or a murine anti-CD62 antibody (KPL1, PL1 or PL2) or in the absence of any antibody (control). FACS analysis was then performed and expression of the early activation marker CD69 on natural killer (NK) cells (CD56+) was determined. As shown in FIG. 8, activation of NK cells by Y1-IgG was mediated with all six donor cells. In contrast, no effect could be detected by either human IgG or by anti-CD162 mouse antibodies KPL1, PL1 or PL2. Preliminary studies have also shown induction of FasL expression on effector cells following incubation with Y1-IgG (data not shown).

e. Y1-IgG Induced Apoptosis

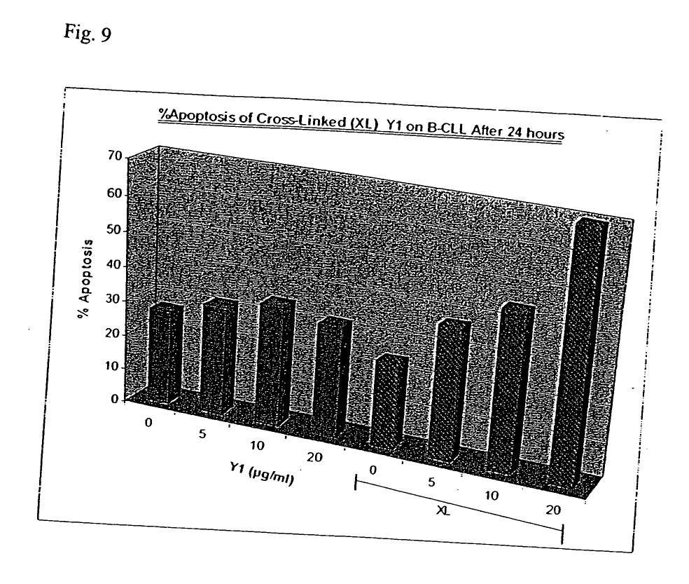

Mononuclear cells (CD 19+, CD5+) from B-CLL patients incubated in the presence of Y1-IgG exhibited about 5% apoptosis within 24 hours, as assessed by FACS analysis (FIG. 9). Addition of secondary antibodies that cross-link the Y1-IgG elicited an additional 50% of apoptosis within 24 hours (FIG. 9).

These results suggest that cross-linking of an antibody directed to a sulfated epitope on PSGL-1 triggers signals for apoptosis of primary B-CLL cells. This implies that PSGL-1 can be a target for inducing apoptosis in B-CLL patients in vivo, wherein the cross-linking effect may be mediated by Fc receptor bearing cells, e.g. monocytes, CD56+ NK cells and γδ⁺T cells.

The apoptotic and cross-linking effects described above may be inhibited using the anti-PSGL-1 antibody KPL1 (data not shown). This antibody on its own does not induce apoptosis. This provides confirmation that the apoptotic signal is mediated via an epitope on PSGL-1.

f. The ADCC Effect of Y1-IgG on B-CLL Relative to Rituximab

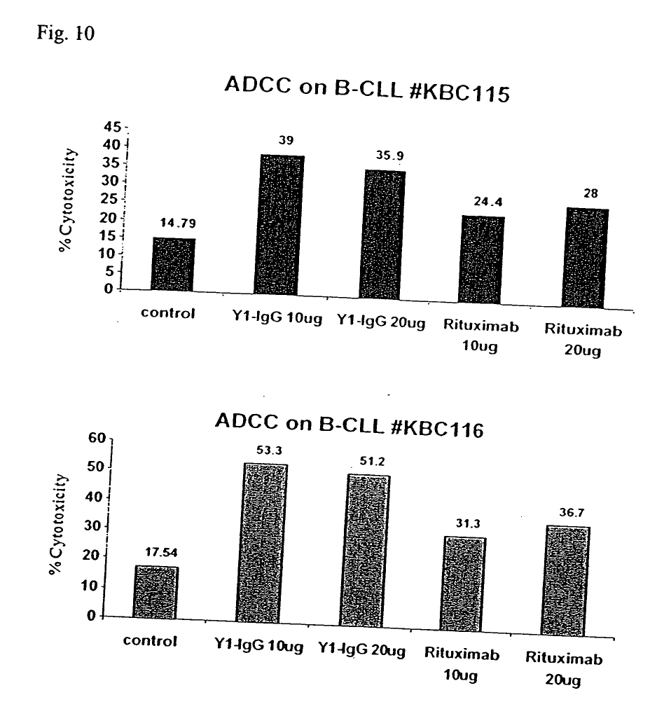

The percentage of cell death induced by the Y1-IgG antibody in two primary human B-CLL patient samples was significantly higher than that obtained by Rituximab. FIG. 10 shows that Y1 induced 25% to 35% cytotoxicity over control compared to only 10% to 13% induced by Rituximab. Saturation of receptor molecules on the target cells was achieved at 10 μg/ml of Y1-IgG antibody but not by the same concentration of Rituximab.

Taken together, the results suggest that Y1-IgG is a promising candidate as a therapeutic agent in the treatment of B-CLL, as it cytotoxic and apoptotic effects appear to be mediated via specific recognition of a PSGL-1 sulfated epitope expressed on these diseased cells.

g. Analysis of the CDC Effect of Y1-IgG, Rituximab and Campath on B-CLL

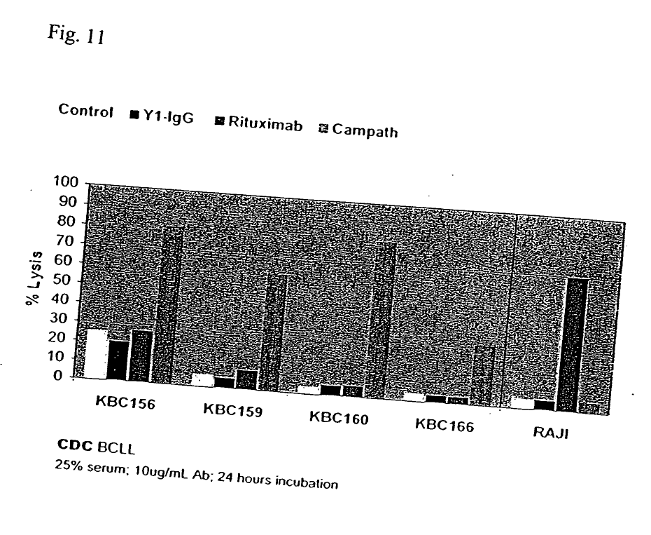

Mononuclear cells from B-CLL patients were incubated with Y1-IgG, Rituximab or Campath® in the presence and absence of 25% of the patients' plasma. As shown in FIG. 11, only Campath® mediated cytotoxicity of primary B-CLL cells via CDC. Neither Rituximab nor Y1-IgG induced cytotoxicity via complement fixation.

These in vitro results indicate that Y1-IgG may be useful as a therapeutic agent for treatment of B-CLL based on its ADCC activity.

### Example 3

### Y1-IgG-M-Daunorubicin Derivative

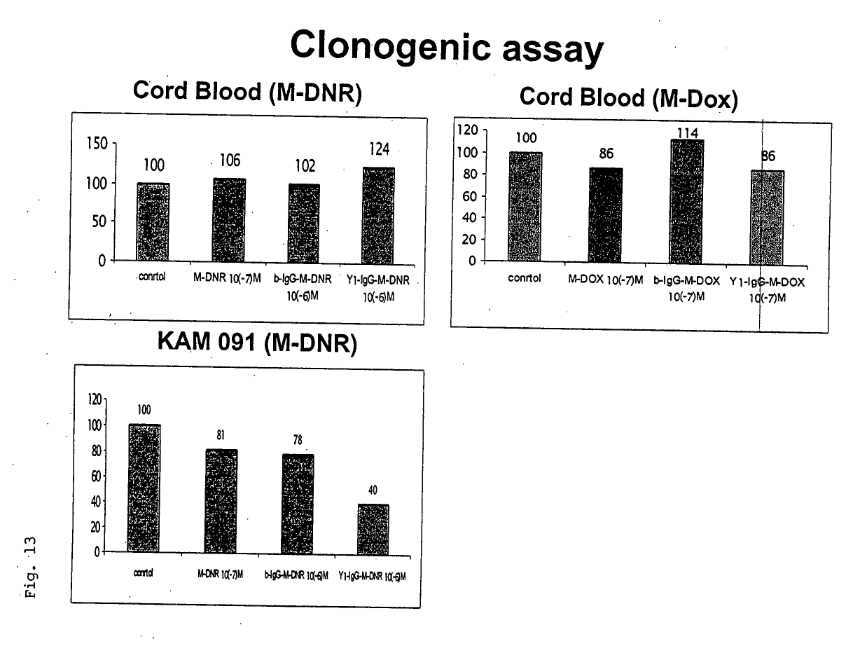

3.1 Preparation of Y1-IgG-M-Daunorubicin Derivative: Antibody-toxin conjugates such as morpholino-doxorubicin-Y1-IgG (FIG. 13) and antibody-M-daunorubicin conjugates (see below) were prepared. Daunorubicin was modified, joined to one of two different linkers, and then joined to the antibody via the antibody's free amino groups or via the antibody's reduced disulfide bonds. The term M-DNR-LINKER refers to both (6-Maleimidocaproyl)hydrazone of Morphlinyldaunorubicin acetate and to M-DNR-AES.

a. Preparation of 3′-Deamino-3′-(4-morpholinyl)daunorubicin acetate (M-DNR-Ac)

Dry triethylamine was added to a solution of daunorubicin hydrochloride in dry dimethylformamide, under argon, followed by bis(2-iodoethyl) ether. The reaction mixture was protected from light and stirred for 36 hours at room temperature.

The resulting aqueous mixture was extracted with methylene chloride. The organic phase was dried over anhydrous sodium sulfate, filtered through celite and evaporated to dryness. The crude product was purified by silica gel column chromatography, and the relevant fractions pooled together and evaporated to yield the M-DNR free base as a red oil, which was found to be 98% pure (by HPLC). The yield was 55%.

After reaction with acetic acid, the resulting free base was isolated as its solid acetate salt followed by lyophilization. M-DNR-Ac is stable for at least 12 months under argon at −20° C.

b. Preparation of (6-Maleimidocaproyl)hydrazone of Morphlinyldaunorubicin acetate

6-maleimidocaproylhydrazide was added to a solution of M-DNR-Ac in dry methanol, under argon, followed by trifluoroacetic acid. The clear solution was protected from light and stirred for 24 hours at room temperature.

The methanolic solution was evaporated to dryness under reduced pressure at 25° C., resulting in a red oily residue, which was dissolved in dry methanol. To this solution, dry ether was added and the precipitated red solid isolated by centrifugation. The pure crystalline product, which had a purity of 98% and a yield of 88%, was obtained after three triturations with dry ether, dried under high vacuum, and kept under argon at −20° C. (6-Maleimidocaproyl)hydrazone of Morphlinyldaunorubicin acetate is stable for at least 4 months under argon at −20° C.

c. Preparation of N-hydroxysuccimimide Ester of Adipic Acid Monohydrazone of Morpholinodaunorubicin (M-DNR-AES)

1. Preparation of Adipic Acid Monohydrazone of Morpholinodaunorubicin

The following were combined and stirred at room temperature under Ar while being protected from light for 1 hour: morpholinodaunorubicin acetate salt, dry MeOH, freshly prepared methanolic solution (hydrazidoadipic acid hydrochloride and Et3N and TFA stock solutions).

The solvent was removed under reduced pressure and the resulting residue dissolved in NH₄OAc:AN and injected into a semiprep HPLC column. The mixture was washed and concentrated under isocratic conditions. The desired product was collected after about 4.5 min and concentrated by C-18 Sep Pak cartridge. The product was eluted, lyophilized, and stored at −20° C. under Ar. The product was obtained as a red solid with an 80% yield and 95% purity.

2. Preparation of N-hydroxysuccimimide Ester of Adipic Acid Monohydrazone of Morpholinodaunorubicin

Hydroxysuccinimide in dry THF and DCC in dry THF were added to the adipic acid monohydrazone of morpholinodaunorubicin in dry THF. The clear, red solution was stirred for 24 h at room temperature under Ar. The end of the reaction was determined by analytical HPLC, and the solvent was removed. The glacial solid was then dissolved in buffer solution (N-methylmorpholinium acetate/AN) and filtered through cotton.

The product was isolated by RP-HPLC, diluted with two volumes of n-Methylmorpholinium acetate solution and loaded on a Sep-Pak (900 mg). The product was eluted and lyophilized to obtain a powder with 73% yield and 97.4% purity.

d. Preparation of M-DNR-Y1-IgG Conjugate

M-DNR-LINKER in dry DMF was added to the MAb solution at a molar ratio M-DNR-LINKER/Y1-IgG of 23. The mixture was gently shaken overnight at room temperature under argon, then centrifuged. The supernatant was filtered through SPIN-X tubes (Costar) and shaken with Bio-Beads SM-2 (Biorad) for 1 hour at room temperature. The mixture was allowed to stand for 10 minutes. The supernatant was passed through PD-10 columns (Pharmacia) that had been equilibrated with PBS. The conjugate was eluted with PBS and the protein-containing fractions combined. The purified conjugate was sterilized by SPIN-X filtration. The conjugate solutions were frozen and stored at −70° C. The product conjugate was obtained in 45-50% yield and had the following characteristics: 5% aggregates; 2%-5% absorbed, non-covalently linked M-DNR derivatives; average molecular ratio of drug to antibody is 4.

e. Preparation of M-DNR-Y1IgG Conjugate (via Reduction of the S—S Bonds in the Fe Region)

Y1-IgG in a buffer composed of NaCl, MES and EDTA was added to cysteamine hydrochloride solution (Merck) in the same buffer. The mixture was incubated at 37° C. for 1.5 hours under argon. The reaction mixture was loaded onto a PD-10 column (Sephadex G-25, Pharmacia) that had been equilibrated with PBS/EDTA. The reduced protein was eluted with PBS/EDTA. The fractions with the highest protein concentrations were combined and stored at 4° C. The molecular ratio of free sulfhydryl groups to antibody was at least 3.5. (6-Maleimidocaproyl)hydrazone of morphlinyldaunorubicine acetate was diluted in DMF and added to the solution of reduced protein and incubated for 30 minutes at 4° C. The reaction mixture was passed through a PD-10 column preequilibrated with PBS, then eluted with PBS. The protein fractions were combined, then sterilized by SPIN-X filtration (Costar). The purified conjugate was aliquoted and stored at −70° C. The product conjugate was obtained in ˜50% yield and had the following characteristics: less than 5% aggregates; 1-2% free M-DNR derivatives; average molecular ratio of drug to antibody of 4.

f. Preparation of IgG-Y1-M-DNR Conjugate (via Reduction of the S—S Bonds)

IgG-Y1 was reduced by passing it through a PD-10 column (Sephadex −25M, Pharmacia) in EDTA and eluting in PBS/EDTA. The protein-containing fractions were pooled. The molecular ratio of free sulfhydryl groups to antibody was at least 6. (6-Maleimidocaproyl)hydrazone of morphlinyldaunorubicin acetate in dry dimethylformamide was diluted in DMF and added to the reduced protein. The solution was incubated for 30 minutes at 4° C. The protein conjugate was purified on a PD-10 column in PBS and the protein fractions were sterilized by SPIN-X filtration (Coming Life Sciences). The conjugate was frozen and stored at −70° C. Yield was about 50% relative to original antibody. The final product contained less than 5% aggregates, free M-DNR was between 0-2%, and the average molecular ratio of drug to antibody was 7.

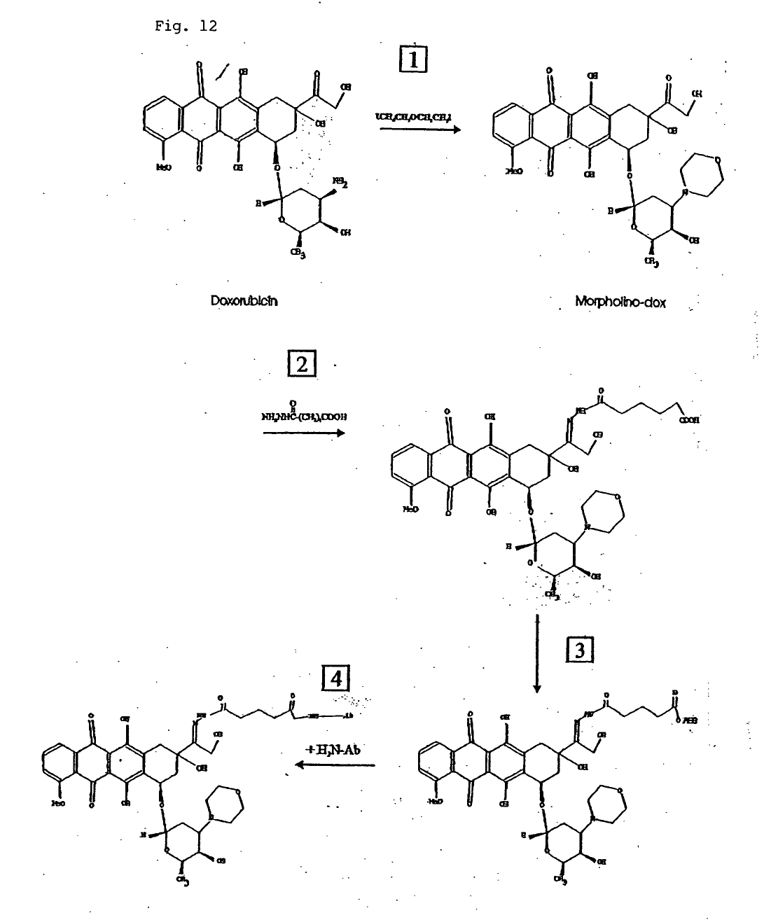

3.2 Cytotoxicity of Y1-IgGM-DNR Derivative (FIG. 12-14): The specific cytotoxic effect of Y1-drug conjugates was assessed in semisolid (METHOCULT™; StemCell Technologies Inc., Vancouver BC, Canada) clonogenic assays. Cells (CD34+ stem cells from cord blood or primary AML patient samples) were incubated with free or conjugated drugs at concentrations of up to 10⁻⁶M for 1 hour at 37° C. The cells were washed, seeded in METHOCULT™ and grown for 10-12 days, after which colonies were counted. Bovine (b)-IgG-M-DNR was used as a non-specific conjugate control.

The results (FIG. 13) showed limited effect of the conjugate Y1-IgG-M-DNR on the cord blood samples. That is, non-target cells (healthy CD34+ stem cells) incubated in the presence of 1 μM Y1-IgG-M-DNR survived at least as well as control cells and cells incubated in the presence of 1 μM b-IgG-M-DNR or 0.1 μM M-DNR (i.e. a non-lethal dose of free drug). Thus, no significant effect was seen in all the cord blood samples.

In contrast, in an AML sample (M7 megakaryocytic leukemia), Y1-IgG M-DNR was three times more inhibitory in inhibiting colony growth, relative to the non-specific b-IgG-M-DNR conjugate at the same concentration (FIG. 13). That is, 1 μM Y1-IgG-M-DNR reduced viability of primary AML cells to 40% relative to the control (target cells incubated alone), while 0.1 μM M-DNR and 1 μM b-IgG-M-DNR each reduced viability of primary AML cells to 80% relative to the control.

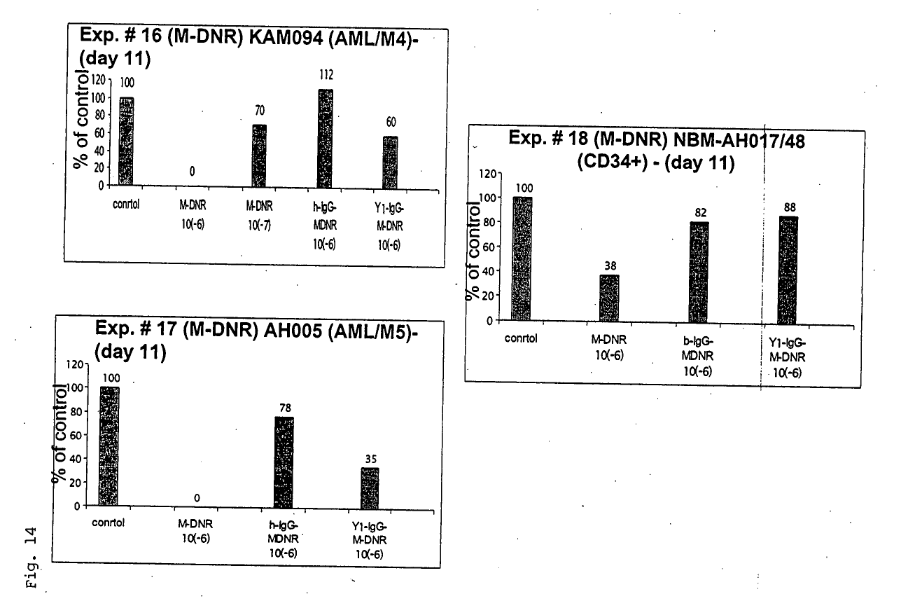

The specificity of the Y1-IgG-M-DNR conjugate was further demonstrated in a similar experiment showing that following incubation with 1 μM Y1-IgG-M-DNR, colony formation from primary AML-M4 and AML-M5 cells (obtained from patients) was inhibited to 60% and 35% respectively, relative to control (FIG. 14). In contrast, all AML-M4 cells and 78% of AML-M5 cells gave rise to colonies following incubation with 1 μM h-IgG-M-DNR. Cells incubated with 1 μM M-DNR did not give rise to colonies, confirming that the cells were sensitive to the drug.

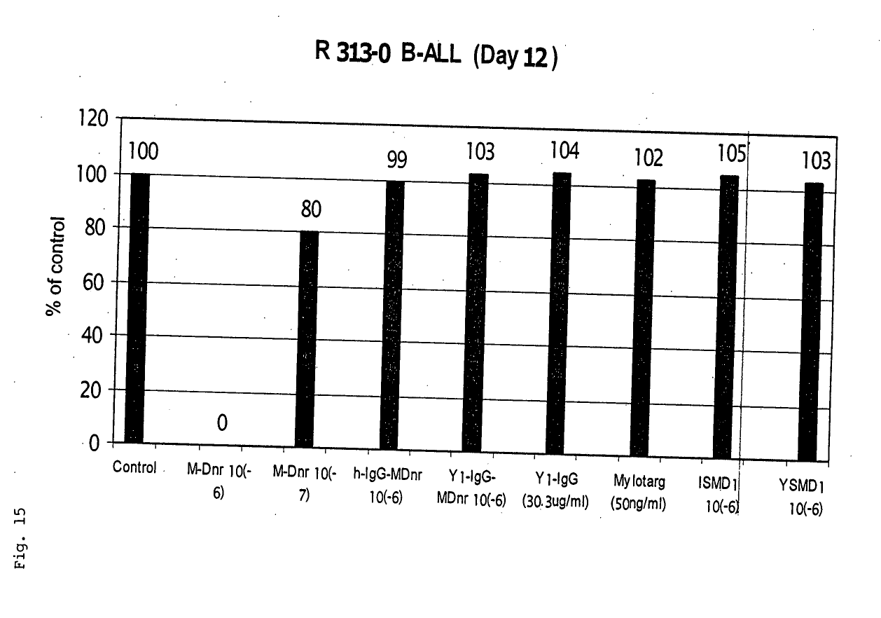

B-ALL cells, which do not express a Y1 epitope (as assessed by failure to bind Y1), are not sensitive to Y1-IgG-M-DNR and thus gave rise to colonies following incubation with 1 μM Y1-IgG-M-DNR at the same rate as the control (FIG. 15).

### Example 4

### Y1 Recognition Motif and Sulfation

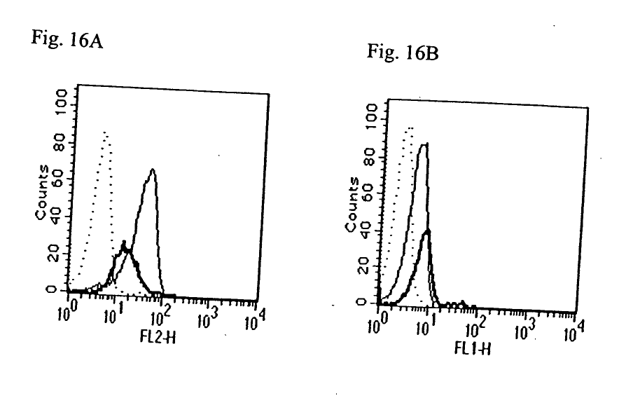

To evaluate the potential effect of tyrosine sulfation on the binding of Y1-scFv to GPIb, the human chronic myeloid leukemia cell line KU812 expressing GPIb was grown in sulfate-free medium in the presence of 100 mM sodium chlorate to inhibit sulfation. Under the conditions employed, tyrosine sulfation is inhibited by up to 95% without affecting protein synthesis or other post-translational modifications. As show in FIG. 16A, binding of Y1-scFv to KU812 cells was reduced by 50% following growth with sodium chlorate, as compared to control cells grown in complete medium lacking sodium chlorate. Under the same conditions, surface expression of GPIb on sulfate-starved cells was unchanged, as indicated by FACS analysis using the mouse anti-GPIb monoclonal antibody AK2-FITC (FIG. 16B).

To further evaluate whether tyrosine sulfate modification plays a role in Y1-scFv binding to GPIb, various synthetic peptides based on residues 273-285 of GPIb were assessed for the ability to inhibit binding of Y1-scFv to platelets.

Briefly, peptides were synthesized by solid phase methodology using an ABIMED AMS-422 multiple peptide synthesizer, and as required, tyrosine sulfate was incorporated using FMOC-Try sodium salt. Synthetic peptides were purified using a Lichrosorb RP-18 column. For the Y1-scFv platelet binding assay, a mixture of synthetic peptide (2.5, 25 or 200 μM) and Y1-scFv (10 μg) was incubated with washed. Following washing, platelets were incubated with R-phycoerythrin labeled-anti scFv, washed and analyzed by FACS.

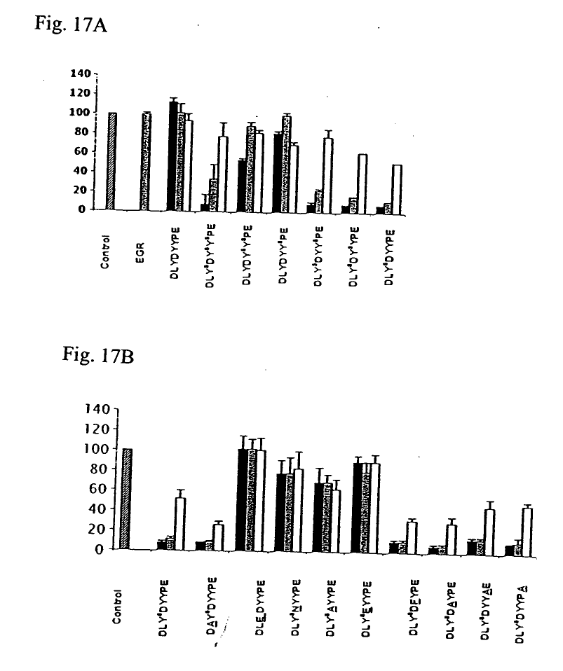

As shown in FIG. 17A, peptide DLY^{S}DY^{S}Y^{S}PE (SEQ ID NO:4) (comprising 3 sulfated tyrosines) at 25 μM effectively inhibited binding Y1-scFv to platelets, while the corresponding non-sulfated control DLYDYYPE (SEQ ID NO:5), had no effect even at 200 μM. Furthermore, each of peptides DLY^{S}DYYPE (SEQ ID NO:6), DLY^{S}DY^{S}YPE (SEQ ID NO:7) and DLY^{S}DYY^{S}PE (SEQ ID NO:8) (sulfated at the first, the first and second, and the first and third tyrosines, respectively) at 25 μM effectively inhibited Y1-scFv binding. Peptides DLYDY^{S}Y^{S}PE (SEQ ID NO:9) and DLYDYY^{S}PE (SEQ ID NO:10) (sulfated at the second and third, and third tyrosines, respectively) had no effect on Y1-scFv binding even at 200 μM (FIG. 17A). These results clearly demonstrate that sulfation at Tyr-276 in GPIb, i.e. the “first” tyrosine position, is important for significant competition and therefore for Y1-scFv binding to GPIb.

To assess whether additional amino acids within the region of residues 273-285 of GPIb contribute to Y1-scFv binding, substitution mutant peptides based on the peptide DLY^{S}DYYPE were tested in the Y1-scFv platelet binding assay (FIG. 17B). When sulfated Tyr-276 was replaced with a negatively charged Glu residue, the mutant peptide DLEDYYPE (SEQ ID NO:11) exerted no substantial inhibition on Y1-scFv binding to platelets, in contrast to DLY^{S}DYYPE. Similarly, mutant peptides DLY^{S}EYYPE (SEQ ID NO: 12), DLY^{S}NYYPE (SEQ ID NO: 13) and DLY^{S}AYYPE(SEQ ID NO:14), having Asp-277 replaced by Glu, Asn, and Ala respectively were substantially incapable of inhibiting Y1-scFv binding to platelets. On the other hand, replacement of Leu-275 by Ala (DAY^{S}DYYPE) (SEQ ID NO: 15)and various replacements of amino acids 278 to 280 [DLY^{S}DFYPE (SEQ ID NO:16), DLY^{S}DAYPE (SEQ ID NO:17), DLY^{S}DYYAE (SEQ ID NO:18) and DLY^{S}DYYPA (SEQ ID NO:19)] yielded mutant peptides which all inhibited Y1-scFv binding to platelets, in a manner substantially identical to that of DLY^{S}DYYPE (FIG. 17B).

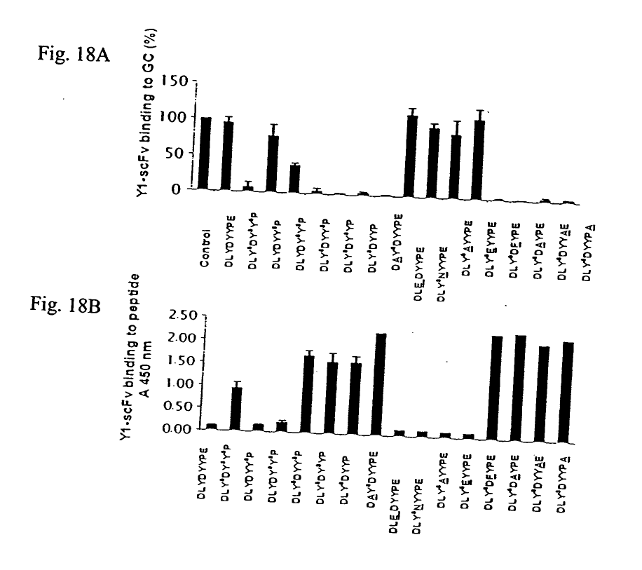

To validate the platelet binding inhibition assay, the mutant peptides were also tested for inhibition of Y1-scFv binding to purified glycocalicin (FIG. 18A). Briefly, glycocalicin immobilized on microtiter plates was incubated with Y1-scFv (5 μg/ml) and peptide (25 μM). Following washing, bound Y1-scFv was detected using polyclonal anti-scFv (generated by immunization of a rabbit with an scFv mixture) and anti-rabbit IgG antibody conjugated to horseradish peroxidase, and reading the absorbance at 450 nm in an ELISA reader.

The results obtained in the glycocalicin binding inhibition assay (FIG. 18A) indicated that both sulfated Tyr-276 and the adjacent residue Asp-277 of GPIb are important for Y1-scFv binding, thus confirming the results obtained in the platelet binding inhibition assay (FIGS. 17A and B). Additional confirmation was obtained by evaluating by ELISA the direct binding of Y1-scFv to peptides covalently coupled to CovaLink Plates (FIG. 18B). This study indicated that GPIb-derived mutant peptides having replacements of sulfated Tyr-276 or of Asp-277 were substantially incapable of direct binding by Y1-scFv, in contrast to mutant peptides having replacements at positions 275, 278, 279 or 280, or having non-sulfated Tyr-278 or Tyr-279, all of which were substantially capable of direct binding by Y1-scFv.

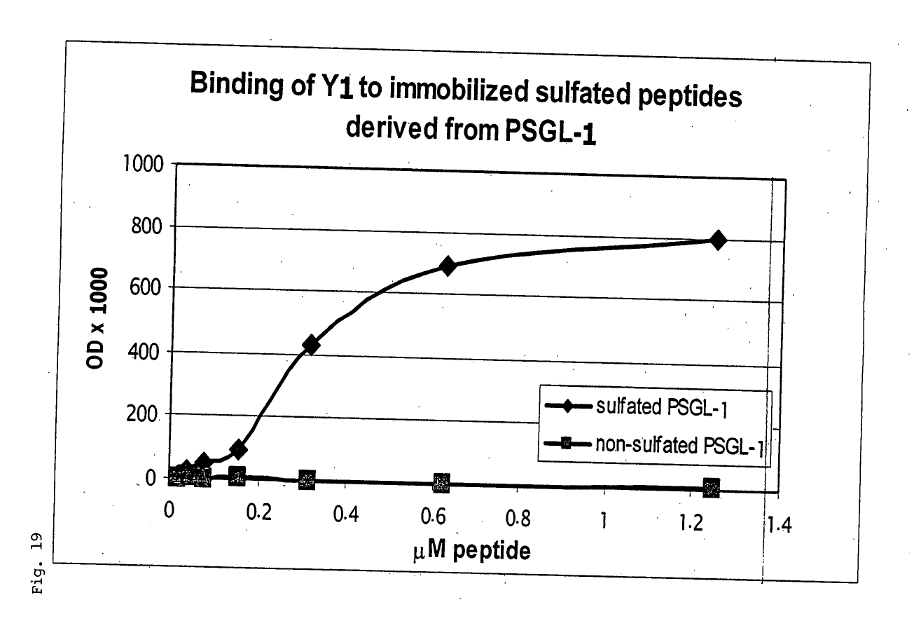

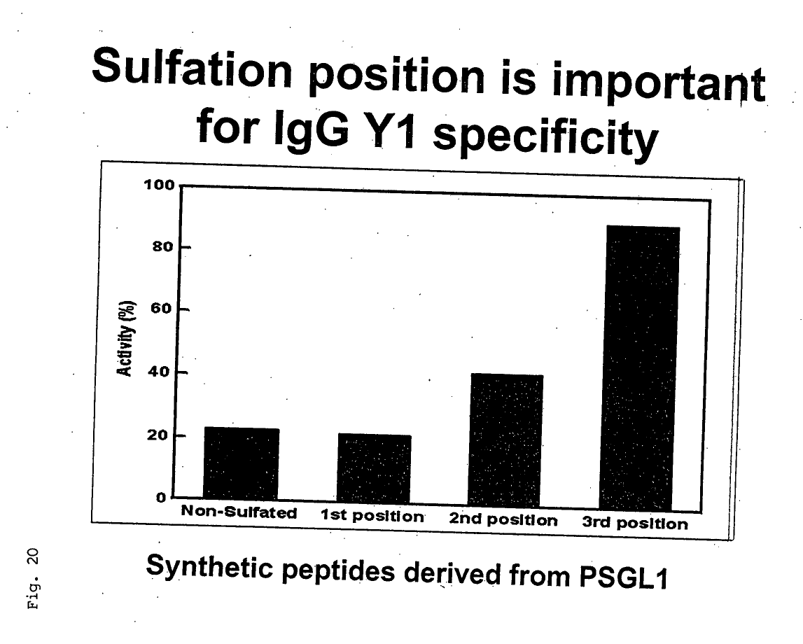

Analogous direct binding experiments using synthetic peptides based on the residues 42-58 of PSGL-1 confirmed that PSGL-1 tyrosine sulfation is important for Y1-scFv binding (see FIG. 19). Specifically, sulfation of the third tyrosine position in the PSGL-1 sequence QATEYEYLDYDFLPETE (SEQ ID NO:20) results in approximately 100% Y1-scFv binding activity relative to the corresponding unsulfated control peptide (see FIG. 20). In contrast, sulfation of the same linear peptide at the second tyrosine position confers only about 40% binding relative to the control, and sulfation at the first tyrosine position confers binding activity substantially indistinguishable from the control.

Taken together, the results obtained using synthetic peptides based on GPIb and PSGL-1 indicate that the sequence Y^{S}D, which is found within the motif DXY^{S}D (SEQ ID NO:21), wherein X represents any amino acid and Y^{S} represents sulfated tyrosine, is important for Y1 binding to its epitope.

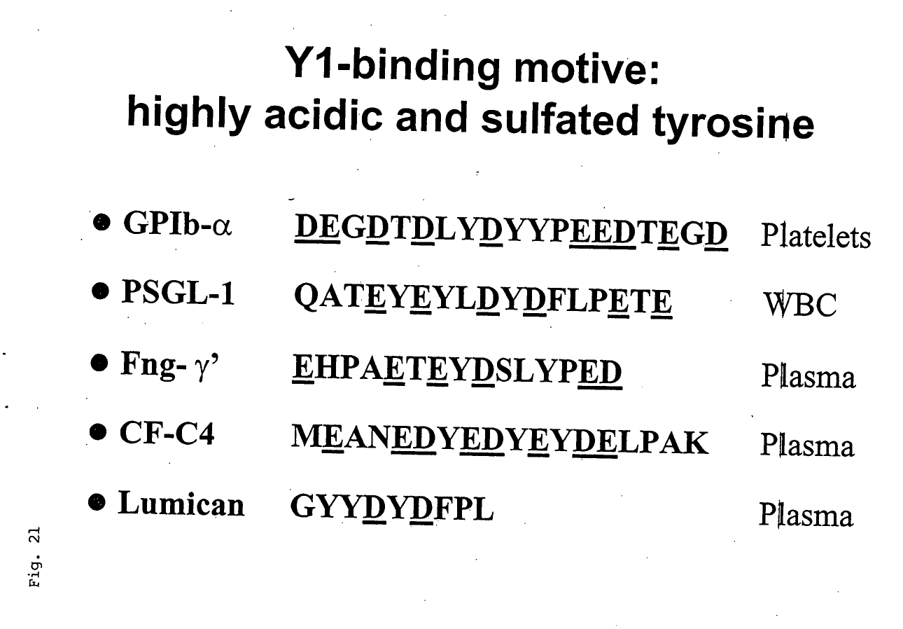

Several proteins are known to contain the sequence Y^{S}D found within the motif DXY^{S}D and/or within a highly acidic environment (FIG. 21). It is believed that such an acidic environment is significant for tyrosine sulfation in vivo. It is predicted that Y1 is capable of binding such proteins at this sulfated sequence, as has been shown for GPIb and PSGL-1.

4.2 Y1 Binds to Solid Tumor Antigens While KPL-1 Does Not:

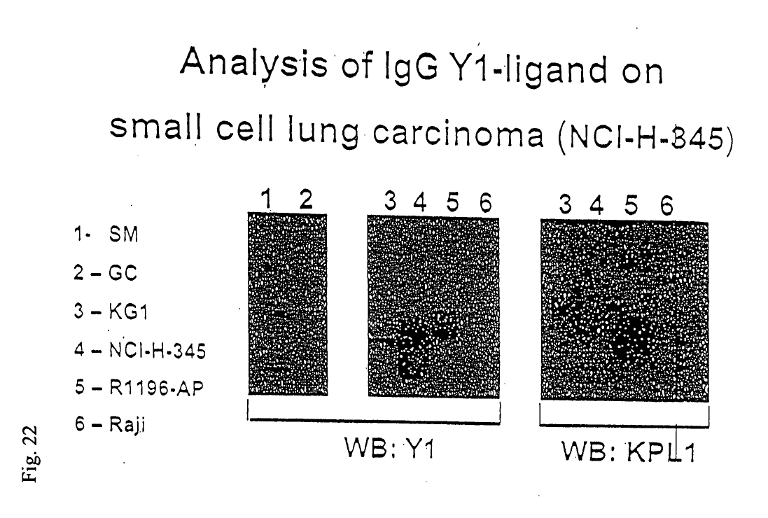

Western blotting of a small cell lung carcinoma (SCLC) cell line lysate was performed to compare binding of Y1 and the commercially available mouse anti-PSGL-1 antibody KPL1 (FIG. 22). A single broad band was observed in the Y1 blot, whereas no band was observed in the KPL1 blot. This indicates that PSGL-1 may serve as a target for immunotherapy in SCLC patients using Y1.

### Example 5

### Y1-IgG-Mediated Endocytosis via PSGL-1

PSGL-1 is highly expressed on AML patients' blood cells. The results below indicate that Y1 specifically recognizes and is internalized by tumor cells expressing PSGL-1. Commercially available KPL-1 (anti-PSGL-1) binds to tumor cells but is not internalized. This indicates that PSGL-1 may serve as a target for immunotherapy in AML patients using Y1.

5.1 Confocal Microscopy Studies: Patients' white blood cells were isolated on FICOLL® gradient. Cells were incubated for different time periods, at 37° C., in the presence of fluorescent anti-PSGL-1 antibodies (Y1 or commercial antibodies KPL1 and PL1). Fluorescent antibody localization was determined in live cells by visualizing the cells by confocal microscopy.

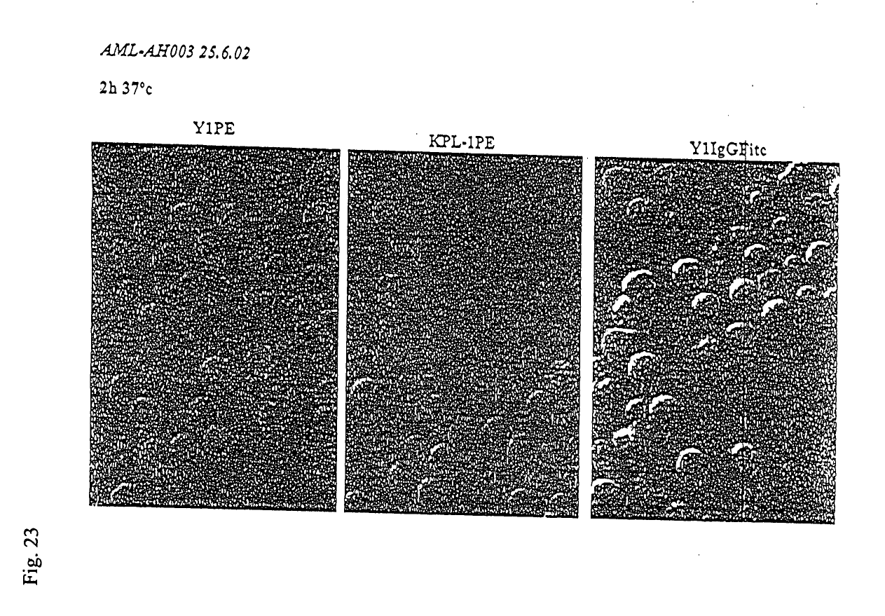

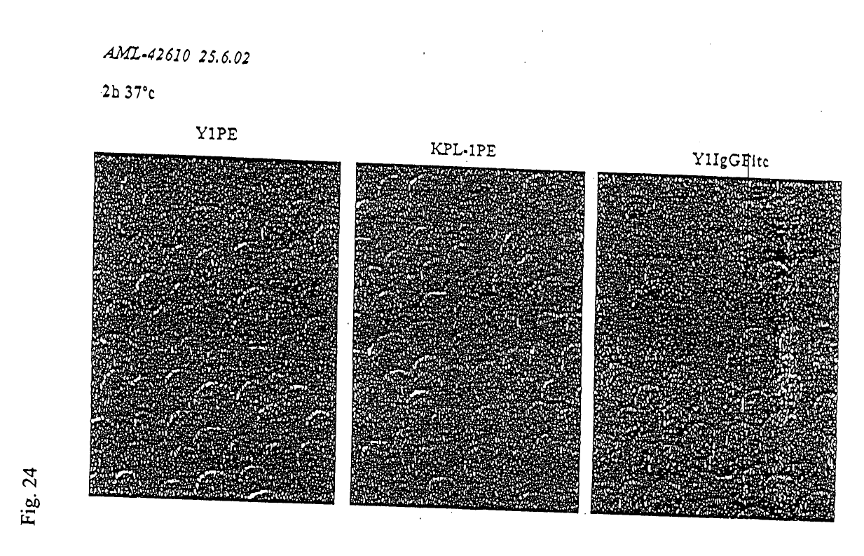

FIGS. 23 and 24 show live AML patient's cells visualized by confocal microscopy following incubation of the cells at 37° C. for 2 hours with Y1-PE (left), KPL1-PE (middle) and Y1-IgG-FITC (right). Cells were scanned in three-dimensional manner (X, Y and Z plans) and the pictures presented here were taken from the center of the sphere in respect to the Z plan. As shown, following incubation Y1-IgG was present in the interior part of the cells (but not in the nucleus), while KPL1 was present on the cell membranes and did not internalize.

5.1 Fluorescence Microscopy Studies: Patients' white blood cells were isolated on FICOLL gradient. Cells were incubated for different time periods, at 37° C., in the presence of Y1-IgG. For detection of the Y1-IgG, cells were fixed, permeabilized and then stained with rhodamine-labeled anti-human (Fc) antibodies. The cells were visualized by fluorescence microscopy.

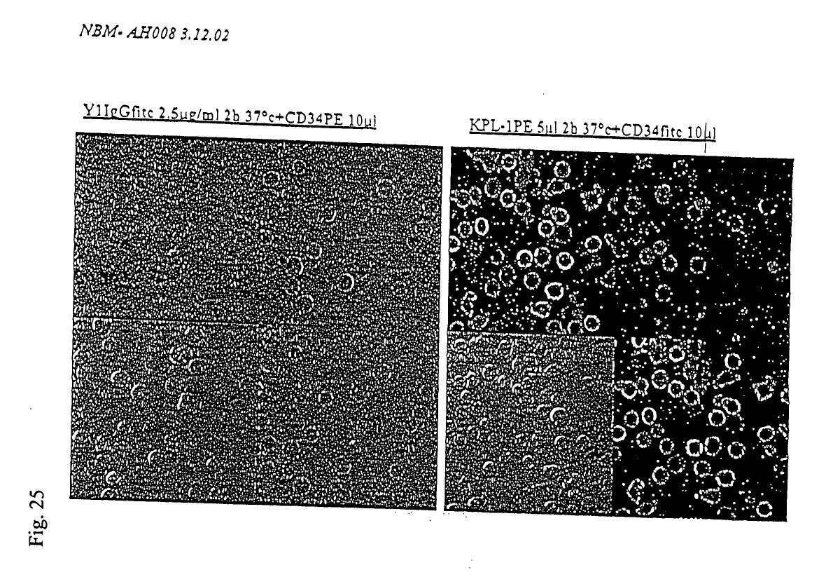

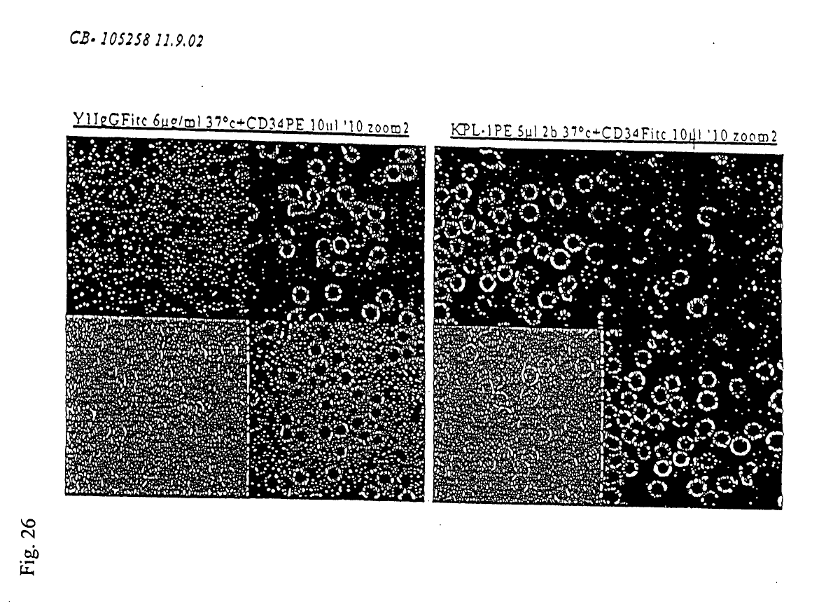

FIGS. 25 and 26 show live CD34+ cells (from healthy bone marrow and from healthy cord blood, respectively) visualized by Confocal Microscopy following incubation of the cells at 37° C. for 2 hours with Y1-IgG-FITC (left) and KPL1-PE (right) and with anti-CD34-PE or FITC. As shown, Y1-IgG did not bind normal CD34+ stem cells. In contrast, KPL-1-PE labeled normal cells including CD34+ cells, as evidenced by double staining of some cells in the lower right panel.

5.3 Monitoring Endocytosis: Cell surface binding of Y1-IgG was detected after incubation of the cells in the presence of the antibody, at 4° C. with 0.1% NaN₃ (which inhibits active process of internalization). Incubation at 37° C. for 10 min-2 hrs (without NaN₃) resulted in capping and patching and in internalized staining. Similar pictures were obtained with different AML samples either by means of confocal microscopy or by means of fluorescence microscopy.

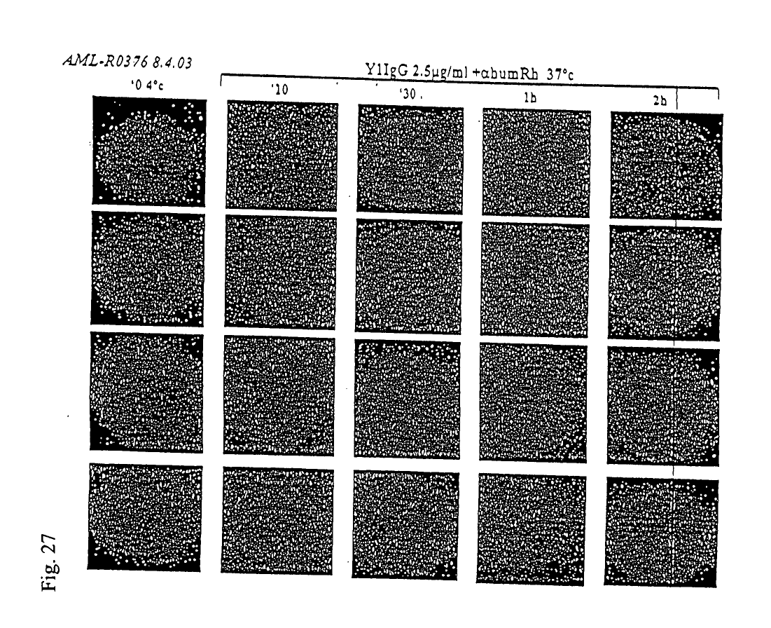

FIG. 27 shows four individual cells from an AML patient sample incubated with non-labeled Y1-IgG for different time periods, at 37° C. or at 4° C. As shown, cell surface binding of Y1-IgG was detected after incubation of the cells in the presence of the antibody, at 4° C. with 0.1% NaN3. Incubation of Y1-IgG at 37° C. for 10 minutes-2hours (without NaN₃) resulted in capping and patching and in internalized staining. Internalization increased with time. No internalization was observed in cells kept at 4° C. Y1-IgG was detected with rhodamine-labeled anti-human (Fc) antibodies. Visualizing the cells was performed by fluorescence microscopy.

5.4 Acid Stripping: Treatment of the cells with 50 mM Glycine (pH 2.5) resulted in removing of cell surface binding of Y1-IgG and enabled detection of internalized Y1-IgG.

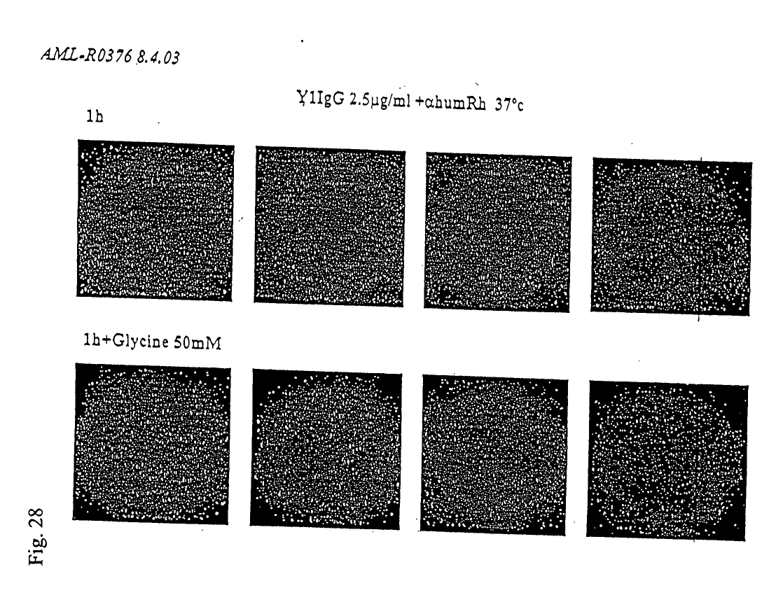

FIG. 28 shows AML patient's cells that were incubated with non-labeled Y1-IgG for 1 hour, at 37° C. Cells shown in the lower row were then incubated at room temperature for 5 minutes with 50 mM glycine, pH 2.5 to remove surface bound Y1-IgG. As shown, the upper panel represents cell surface capping and patching and internalized Y1-IgG. In the lower panel, only internalized Y1-IgG was detected.

5.5 Pronase: Removing of cell surface proteins by the proteolytic enzyme, pronase, also resulted in removing of cell surface binding of Y1-IgG and enabled detection of internalized Y1-IgG.

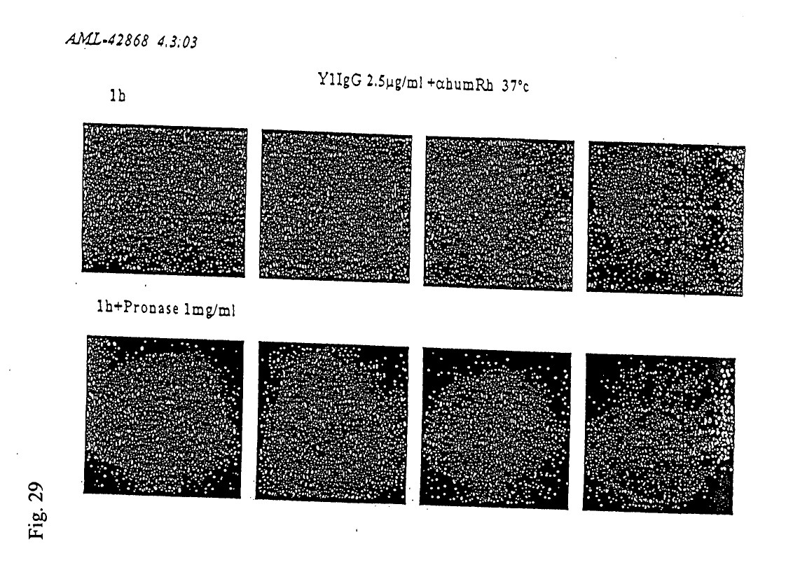

FIG. 29 shows AML patient's cells that were incubated with non-labeled Y1-IgG for 1 hour at 37° C. Cells shown in the lower row were then incubated at room temperature for 60 minutes with 1 mg/ml pronase to remove surface bound Y1-IgG. As shown, the upper panel represents cell surface capping and patching and internalized Y1-IgG. In the lower panel, only internalized Y1-IgG is detected. Note that the uropods were removed by pronase, which implies that the uropods formed by Y1-IgG cross-linking of CD 162 are formed on the outer surface of the cell surface.

5.6 Coated-pits mediated endocytosis: Receptor mediated endocytosis can occur via coated-pits. Coated-pits-mediated endocytosis can be blocked by incubation of the cells with 0.45M sucrose for 15 minutes at 37° C. prior to incubation with Y1-IgG. This method inhibited endocytosis of Y1-IgG without affecting binding of Y1-IgG to the cell surface.

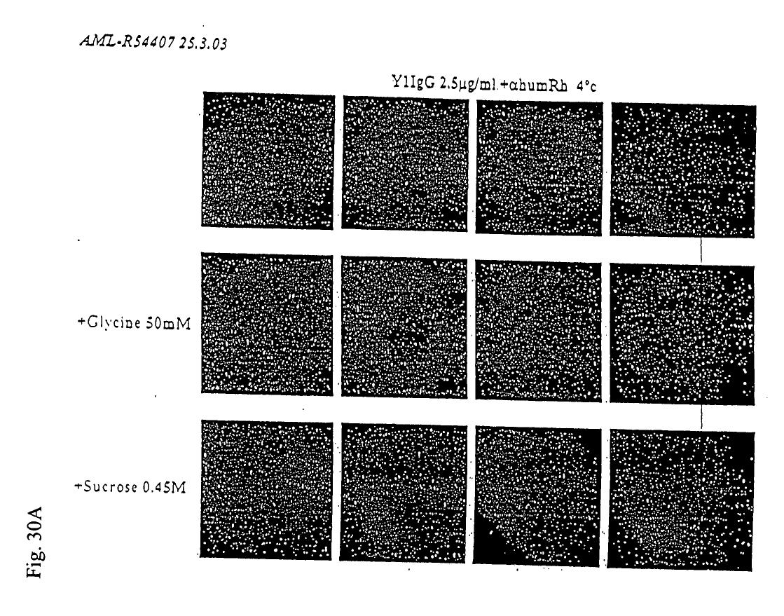

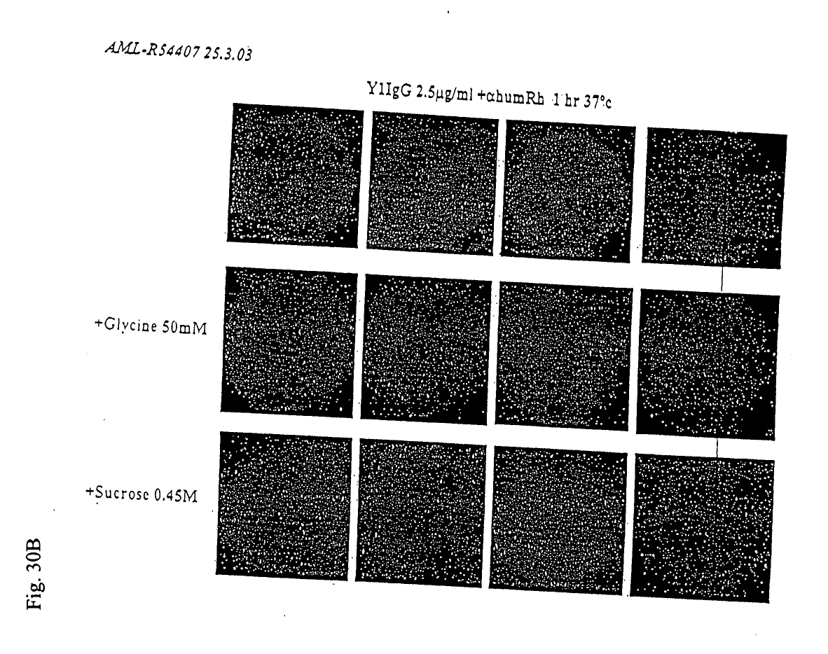

FIG. 30 shows AML patient's cells that were incubated with non-labeled Y1-IgG for 1 hr, at 4° C. (FIG. 30A) or at 37° C. (FIG. 30B). Cells shown in the middle row were then incubated at room temperature for 5 minutes with 50 mM Glycine pH 2.5 to remove surface bound Y1-IgG. As shown (FIG. 30A), the upper panel represents cell surface staining of Y1-IgG. The surface bound Y1-IgG was removed by the acid wash (middle panel). At 37° C. (FIG. 30B) capping and patching and internalization of Y1-IgG was observed (upper panel). Acid wash removed cell surface bound Y-IgG and only the internalized antibody could be detected (middle panel).

Blocking of coated-pits mediated endocytosis (bottom rows) was obtained by incubation of the cells with 0.45M Sucrose for 15 minutes at 37° C. prior to incubation with Y1-IgG. As shown in the lower panels, treating the cell with 0.45M sucrose did not affect binding of Y1-IgG to the cells at 4° C. (FIG. 30A) but inhibited internalization at 37° C. (FIG. 30B).

### Example 6

### Y1-IgG-Inhibition of Leukocyte-Platelet Interactions

Adherence of leukocytes to vascular surfaces results in organ injury in various disorders, including reperfusion injury, stroke, mesentaric and peripheral vascular disease; organ transplantation and circulatory shock. Reperfusion injury is associated with adherence of leukocytes to vascular endothelium in the ischemic zone, presumably in part due to activation of platelets and endothelium by thrombin and cytokines, which renders their surfaces adhesive for leukocytes. The main initiator of reperfusion injury is the interaction between von Willbrand factor (vWF) and platelet GPIb receptor. Cardiac patients who are treated with thrombolytic agents such as tissue plasminogen activator and streptokinase to relieve coronary artery obstruction, may still suffer myocardial necrosis due to reperfusion injury. Thus, there is a need for drugs which are capable of reducing leukocyte adherence to vascular surfaces and which may be administered in conjunction with thrombolytic agents to improve outcome of cardiovascular disorders.

Since Y1 (both scFv and full IgG) binds to distinct sulfated molecules on platelets (i.e. GPIb) and leukocytes (i.e. PSGL-1), this antibody has potential as a therapeutic agent for inhibiting various cell-cell interactions.

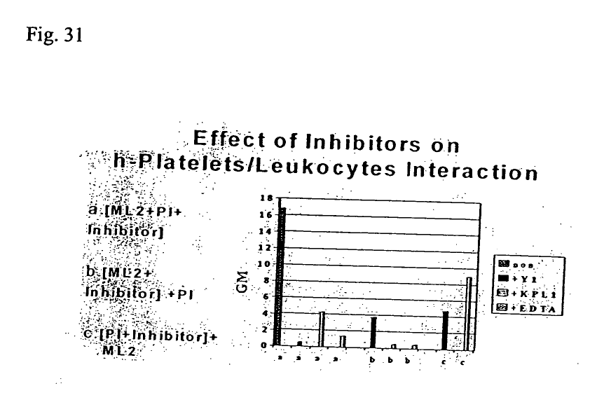

FIG. 31 shows that Y1-scFv effectively inhibits the binding of activated human platelets to ML2 cells (a human AML derived cell line expressing PSGL-1). Optimal inhibition was obtained when the antibody was incubated simultaneously with both platelets and ML2 cells, while partial inhibition was obtained when the antibody was initially incubated with either platelets or ML2 cells, followed by removal of non-bound antibody and subsequent addition of the remaining cell type (FIG. 31).

FIG. 31 also shows that the murine antibody KPL1 (directed against human PSGL-1 N-terminal domain, but not tyrosine sulfation dependent) was also effective in inhibiting binding of activated platelets to ML2, but the inhibition was less than that exerted by Y1-scFv. This might be due to the fact that KPL1 does not recognize an epitope present on both cell types, as does Y1-scFv. No inhibition was observed with the murine antibody, PL2 antibody which is also directed against human PSGL-1 (not shown).

### Example 7

### Y1-IgG-Inhibition of Cell Rolling on Immobilized P-Selectin Under Flow Conditions

For preparation of a ligand-coated substrate, recombinant human (rh)-P-Selectin (R&D Systems, Minneapolis, Minn.) was diluted to 0.2-1.0 μg/ml in coating medium (PBS supplemented with 20 mM bicarbonate, pH 8.5) and immediately adsorbed onto a polystyrene plate overnight at 4° C., followed by washing with PBS containing 2 μg/ml human serum albumin (Calbiochem) at 4° C. for 1 hr.

For laminar flow assays a polystyrene plate on which purified ligand was immobilized was assembled in a parallel plate laminar flow chamber as described previously (Lawrence & Springer, Cell 65, 859-873 (1991)). Human neutrophils (isolated from anti-coagulated blood by dextran sedimentation and density separation over FICOLL) or ML-2 cells were washed in H/H medium (Hanks' balanced salt solution, 10 mM HEPES, resuspended in cell binding medium (H/H medium supplemented with 2 mM CaCl₂) at 2×10⁶ cells/ml, and perfused at room temperature through the flow chamber at a rate generating wall shear stress at the desired flow rate, generated with an automated syringe pump (Harvard Apparatus, Natick, Mass.). Upon reaching the upstream side of the test adhesive substrate, the flow rate was elevated to generate a shear stress of 1 dyn/cm², and all cellular interactions were visualized at two different fields of view (each 0.17 mm² in area) using a 10× objective of an inverted phase contrast microscope (Diaphot 300, Nikon Inc., Tokyo, Japan). An imaging system was used for analysis of instantaneous velocities of leukocytes, WSCAN-Array-3 (Galai, Migdal-Ha'emek, Israel) as described previously (Dwir et al., J. Biol. Chem. 275, 18682-18691 (2000)).

Accumulation of rolling leukocytes on the test fields was determined by computerized cell motion tracking. The frequency of rolling cells was defined as the number of cells out of the cell flux that initiated persistent rolling on the adhesive substrate lasting at least 3 s after initial tethering. Cells were incubated with antibodies at different concentrations and perfused to the flow chamber with binding medium containing the same concentration of antibody. Cell rolling was analyzed either following washing of the reagent (“washed”) or in the presence of the reagent.

For image analysis, an imaging system was developed for quantitative analysis of instantaneous velocities of cell rolling on different adhesive substrates. Video frame images consisting of 768×574 pixels (with a pixel size of 1.15 μm using a 10× objective), were digitized using a Matrox Pulsar frame grabber (Matrox Graphics Inc., Dorval, Quebec, Canada), and images were scanned and processed by the WSCAN-Array-3 imaging software (Galai, Migdal-Ha'emek, Israel), running on an Atlas pentium MMX-200 work station. Cell motions were identified from images tracked at 0.02-s intervals. The program output provided the co-ordinates of the center point of each cell in successive interlaced fields at 0.02 s apart.

A computer program for cell motion analysis was developed in collaboration with the laboratory of Professor David Malah (Electric Engineering Faculty, Technion, Haifa, Israel). The software runs under Matlab 5.2 and compares instantaneous positions of individual cells at successive video images over a period of up to 5 seconds. Tethers of individual cells rolling persistently on the ligand-coated field or moving through it in a jerky motion were determined according to changes in instantaneous cell velocities in the flow direction. A rolling pause was defined as an instantaneous velocity drop to below 29 μm/s at shear stresses of 1-1.75 dyn/cm². This threshold velocity value gave optimal correlation between pause analysis performed on representative cells by the computerized system and manually, directly from the video monitor. The step distances between successive pauses of an individual rolling cell were averaged to yield the mean step distance of a given rolling cell.

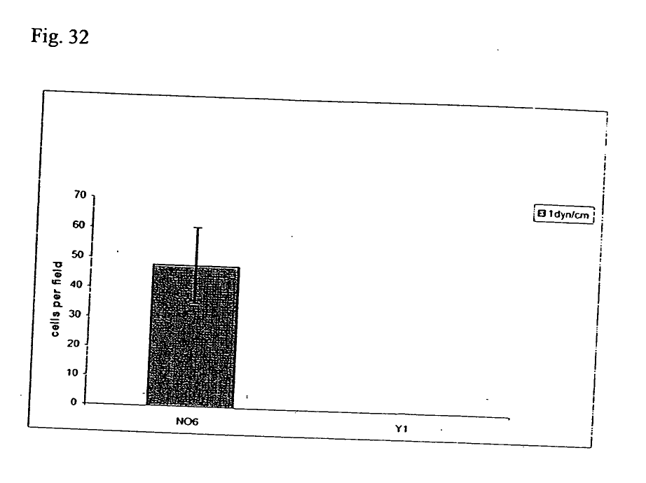

FIG. 32 shows the effect of Y1-scFv (10 μg/ml) on ML2 cell rolling on immobilized rh-P-Selectin at low density (0.2 μg/ml). The analysis showed that at shear force of 1 dyn/cm², the number of rolling cells per field was totally eradicated in the presence of Y1-scFv. No such effect was obtained when equal amounts of the scFv-N06 (negative control) was used.

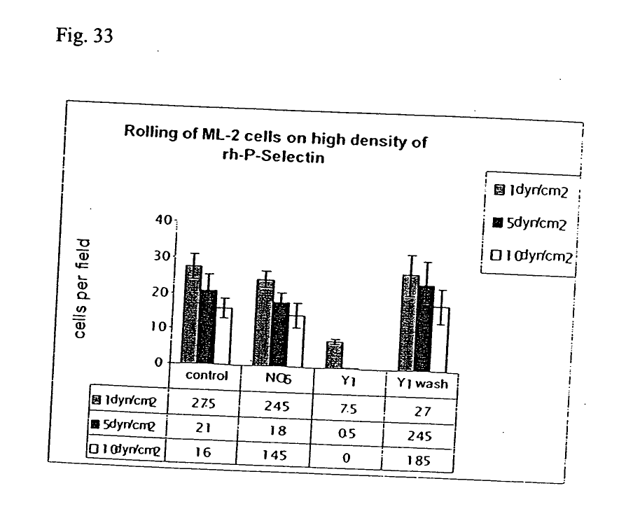

FIG. 33 shows the effect of Y1-scFv (10 μg/ml) on ML2 cell rolling on immobilized rh-P-Selectin at high density (1.0 μg/ml) at various shear forces. The analysis showed that at shear forces of 1, 5 and 10 dyn/cm², the number of rolling cells per field was inhibited by 83%, 98% and 100%, respectively in the presence of Y1-scFv. No such effect was obtained with the negative control, N06, or when cells were washed following incubation with Y1-scFv and then tested (Y1 wash).

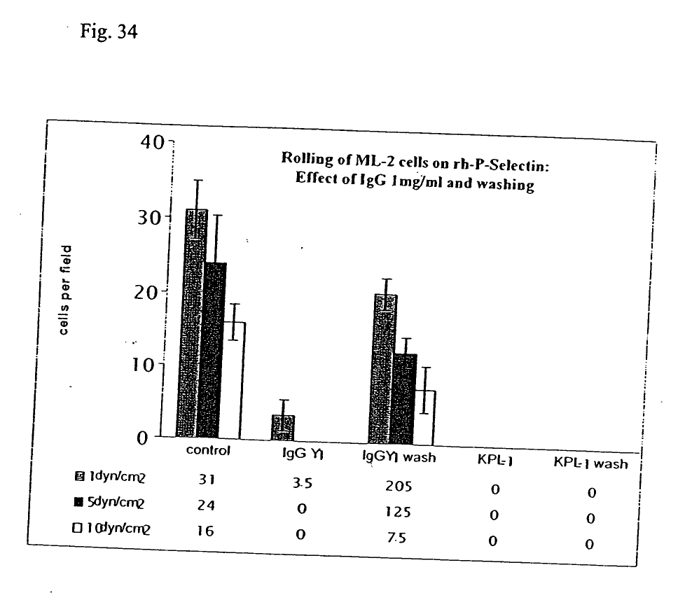

FIG. 34 shows the effect of Y1-IgG (1 μg/ml) on ML2 cell rolling on immobilized rh-P-Selectin (1 μg/ml) at various shear stress forces. The analysis showed that at shear force of 1 dyn/cm², cell rolling was inhibited by 89%, and that at shear forces of 5 and 10 dyn/cm² cell rolling was inhibited by 100%. When cells were washed following incubation with Y1-IgG and then tested (Y1-IgG wash), cell rolling was inhibited by 46%, 48% and 54% at shear forces 1, 5 and 10 dyn/cm², respectively. The murine anti-PSGL-1 antibody KPL1 was also capable of 100% inhibition of cell rolling at all shear forces.

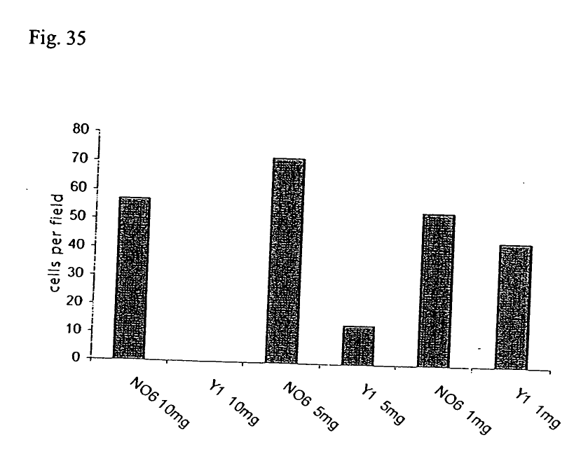

FIG. 35 shows the effect of increasing concentrations of Y1-scFv on human neutrophil rolling on immobilized rh-P-Selectin at high density (1.0 μg/ml). The analysis showed that at a shear force 1 dyn/cm², Y1-scFv at 1, 5 and 10 μg/ml inhibited the number of rolling neutrophils by 20%, 81% and 100%, respectively.

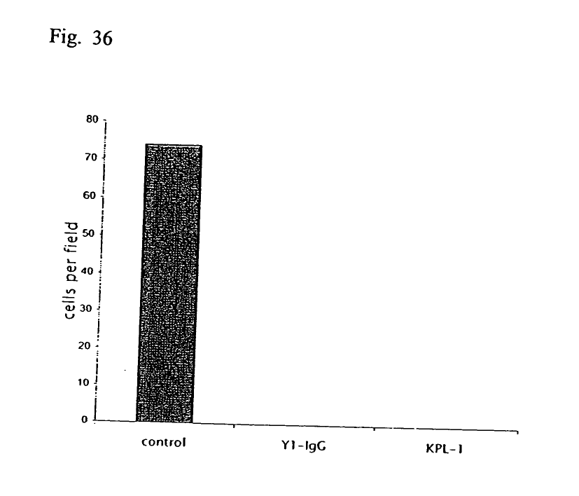

FIG. 36 shows the effect of Y1-IgG on human neutrophil rolling on immobilized rh-P-Selectin at high density (1.0 μg/ml). The analysis showed that at a shear force 1 dyn/cm², the number of rolling neutrophils per field was totally eradicated (100% inhibition) in the presence of Y1-IgG (1 μg/ml). Similar results were obtained with KPL-1.

### Example 8

### Screening of Inorganic Compound Library

Synthetic sulfated peptide (sulfated on a given specific tyrosine residue within the known amino acid sequence of the peptide) derived from a specific receptor (protein) can be prepared with a biotin tag (biotinylated) coupled to the synthetic peptide via a short linker such as caproic acid. Control peptides using the same synthetic peptide can be prepared without sulfation and without the biotin tag (“B”). In addition, synthetic sulfated peptides derived from other, non-related proteins can be prepared without having the biotin tag (“C”) as additional controls.

The biotinylated peptide above (“A”) can be coupled to strepavidin-coated magnetic beads and excess unbound biotinylated peptide then washed away. The biotin-stretavidin peptide conjugate (“D”) can be screened against a small chemical entity library in the presence of large excess of non-sulfated control peptide (“B”) under physiological conditions (37° C., pH 7.0-7.4, salts concentration, conductivity etc.) for molecules that bind to “A”. The coupled-magnetic beads are then washed twice with buffer, each time centrifuged to remove excess unbound molecules. Molecules bound to the magnetic beads (“E”) can be eluted, chemically identified and prepared in larger quantity for further screening.

Confirmation of binding to biotinylated sulfated peptides by the selected chemical compounds (“E”) can be carried out by a further screening process. This process includes either competition with unrelated sulfated peptides that are biotinylated (process 1) or competition with an antibody or fragments thereof (e.g. scFv) that bind specifically to the biotinylated peptide, “A” (process 2).

8.1 Re-screening by Competition with Unrelated Biotinylated Sulfated Peptides (Process 1)

In order to ensure that the compounds bind specifically to “A”, a second round of screening can be carried out. Biotin-streptavidin peptide conjugate (“D”) can be re-screened with the selected compounds “E” in the presence of large excess of unrelated biotinylated sulfated peptides, “C”. The tube is then centrifuged, the biotin-stretavidin peptide conjugate coupled magnetic beads washed twice with buffer and centrifuged each time to remove excess unbound molecules. Compounds that bound to the magnetic beads can be eluted for chemical identification. Larger quantities of the chemical compound can be prepared for further studies, such as validation of selective binding to “A”, and efficacy testing in vitro and in vivo.

8.2 Re-Screening by Competition with Specific scFv Anti-Sulfated Antibody (Process 2)

Compounds with preferred binding affinity to “A” can be re-screened by competing the binding of biotin-streptavidin peptide conjugate (“D”) to each of the selected compounds “E” in the presence of large excess of specific scFv antibody that specifically recognizes and binds to “A”. Chemical compounds that are specifically inhibited from binding to “A” by the scFv antibody can be prepared for further studies, such as validation of selective binding to “A”, and efficacy testing in vitro and in vivo.

The invention has been described with reference to specific examples, materials and data. As one skilled in the art will appreciate, alternate means for using or preparing the various aspects of the invention may be available. Such alternate means are to be construed as included within the intent and spirit of the present invention as defined by the following claims:

1. An antibody or fragment thereof that binds to an epitope comprising the motif D-X-Y-D, wherein X represents any amino acid or the covalent linkage between D and Y, and Y is sulfated.

2. The antibody or fragment thereof of claim 1, wherein the antibody or fragment thereof is complexed with an agent selected from the group consisting of anti-cancer, anti-leukemic, anti-metastasis, anti-neoplastic, anti-disease, anti-adhesion, anti-thrombosis, anti-restenosis, anti-autoimmune, anti-aggregation, anti-bacterial, anti-viral, and anti-inflammatory agents.

3. The antibody or fragment thereof of claim 2, wherein the antibody or fragment thereof is complexed with the agent via free amino groups.

4. The antibody or fragment thereof of claim 2, wherein the antibody or fragment thereof is complexed with between 1 and 16 copies of the agent.

5. The antibody or fragment thereof of claim 4, wherein the antibody or fragment thereof is an IgG, each heavy chain of the antibody or fragment thereof is complexed with about 3 copies of the agent, and each light chain is complexed with about 1 copy of the agent.

6. The antibody or fragment thereof of claim 2, wherein the agent is an anti-viral agent selected from the group consisting of acyclovir, ganciclovir and zidovudine.

7. The antibody or fragment thereof of claim 2, wherein the agent is an anti-thrombosis/anti-restenosis agent selected from the group consisting of cilostazol, dalteparin sodium, reviparin sodium, and aspirin.

8. The antibody or fragment thereof of claim 2, wherein the agent is an anti-inflammatory agent selected from the group consisting of zaltoprofen, pranoprofen, droxicam, acetyl salicylic 17, diclofenac, ibuprofen, dexibuprofen, sulindac, naproxen, amtolmetin, celecoxib, indomethacin, rofecoxib, and nimesulid.

9. The antibody or fragment thereof of claim 2, wherein the agent is an anti-autoimmune agent selected from the group consisting of leflunomide, denileukin diftitox, subreum, WinRho SDF, defibrotide, and cyclophosphamide.

10. The antibody or fragment thereof of claim 2, wherein the agent is an anti-adhesion/anti-aggregation agent selected from the group consisting of limaprost, clorcromene, and hyaluronic acid.

11. The antibody or fragment thereof of claim 2, wherein the agent is selected from the group consisting of toxin, radioisotope, imaging agent and pharmaceutical agent.

12. The antibody or fragment thereof of claim 11, wherein the toxin is selected from the group consisting of gelonin, Pseudomonas exotoxin (PE), PE40, PE38, ricin, and modifications and derivatives thereof.

13. The antibody or fragment thereof of claim 11, wherein the radioisotope is selected from the group consisting of gamma-emitters, positron-emitters, x-ray emitters, beta-emitters, and alpha-emitters.

14. The antibody or fragment thereof of claim 11, wherein the radioisotope is selected from the group consisting of ¹¹¹indium, ¹¹³indium, ^{99m}rhenium, ¹⁰⁵rhenium, ¹⁰¹rhenium, ⁹⁹m technetium, ^{121m}tellurium, 122m tellurium, 125m telluriunm 165-thulium, ¹⁶⁷thulium ¹⁶⁸thulium 123iodine, ¹²⁶iodine, ¹³¹iodine, ¹³³iodine, ^{81m}krypton, ³³xenon, ⁹⁰yttrium, ²¹³bismuth, ⁷⁷bromine, ¹⁸fluorine, ⁹⁵ruthenium, ⁹⁷ruthenium, ¹⁰³ruthenium, ¹⁰⁵ruthenium, ¹⁰⁷mercury, ²⁰³mercury, ⁶⁷gallium and ⁶⁸gallium.

15. The antibody or fragment thereof of claim 11, wherein the pharmaceutical agent is an anthracycline.

16. The antibody or fragment thereof of claim 15, wherein the anthracycline is selected from the group consisting of doxorubicin, daunorubicin, idarubicin, detorubicin, carminomycin, epirubicin, esorubicin, morpholinodoxorubicin, morpholinodaunorubicin, and methoxymorpholinyldoxorubicin.

17. The antibody or fragment thereof of claim 11, wherein the pharmaceutical agent is selected from the group consisting of cis-platinum, taxol, calicheamicin, vincristine, cytarabine (Ara-C), cyclophosphamide, prednisone, fludarabine, chlorambucil, interferon alpha, hydroxyurea, temozolomide, thalidomide and bleomycin, and derivatives and combinations thereof.

18. The antibody or fragment thereof of claim 2, wherein the antibody or fragment thereof is complexed with a vehicle or carrier that can be complexed with more than one agent.

19. The antibody or fragment thereof of claim 18, wherein the vehicle or carrier is selected from the group consisting of dextran, lipophilic polymers, HPMA, and liposomes, and derivatives and modifications thereof.

20. A pharmaceutical composition comprising an antibody or fragment thereof of claim 1 and a pharmaceutically acceptable carrier.

21. A diagnostic, prognostic, or staging kit comprising an antibody or fragment thereof of claim 1 and an imaging agent.

22. The diagnostic, prognostic, or staging kit of claim 21, wherein the imaging agent is a radioisotope.

23. A method of inducing antibody-dependent cell cytotoxicity (ADCC) comprising administering to a patient in need thereof the pharmaceutical composition of claim 20.

24. A method of stimulating a natural killer (NK) cell or a T cell comprising administering to a patient in need thereof the pharmaceutical composition of claim 20.

25. A method of inducing cell death comprising administering to a patient in need thereof an antibody or fragment thereof of claim 2, wherein the antibody or fragment thereof complexed to the agent enters the cell and the antibody or fragment thereof is cleaved from the agent, thereby releasing the agent.

26. A method of treating HIV comprising administering to a patient in need thereof a pharmaceutical composition of claim 20.

27. The method of claim 26, wherein the administration prevents entry of HIV.

28. A method of introducing an agent into a cell that expresses an epitope comprising the motif D-X-Y-D, wherein X represents any amino acid or the covalent linkage between D and Y, and Y is sulfated, wherein the method comprises the following steps:
complexing the agent to an antibody or fragment thereof of claim 1 and
administering to the cell the antibody or fragment thereof complexed to the agent.

29. The method of claim 28, wherein the method treats a disease in a patient in need thereof.

30. The method of claim 28, wherein the method treats cell rolling in a patient in need thereof.

31. The method of claim 28, wherein the method treats inflammation in a patient in need thereof.

32. The method of claim 28, wherein the method treats auto-immune disease in a patient in need thereof.

33. The method of claim 28, wherein the method treats metastasis in a patient in need thereof.

34. The method of claim 28, wherein the method treats growth and/or replication of tumor cells in a patient in need thereof.

35. The method of claim 28, wherein the method increases mortality rate of tumor cells in a patient in need thereof

36. The method of claim 28, wherein the method inhibits growth and/or replication of leukemia cells in a patient in need thereof.

37. The method of claim 28, wherein the method increases the mortality rate of leukemia cells in a patient in need thereof.

38. The method of claim 28, wherein the method alters susceptibility of diseased cells to damage by anti-disease agents in a patient in need thereof.

39. The method of claim 28, wherein the method increases susceptibility of tumor cells to damage by anti-cancer agents in a patient in need thereof.

40. The method of claim 28, wherein the method increases the susceptibility of leukemia cells to damage by anti-leukemia agents in a patient in need thereof.

41. The method of claim 28, wherein the method inhibits increase in number of tumor cells in a patient having a tumor.

42. The method of claim 28, wherein the method decreases number of tumor cells in a patient having a tumor.

43. The method of claim 28, wherein the method inhibits increase in number of leukemia cells in a patient having leukemia.

44. The method of claim 28, wherein the method decreases number of leukemia cells in a patient having leukemia.

45. The method of claim 28, wherein the method inhibits platelet aggregation in a patient in need thereof.

46. The method of claim 28, wherein the method inhibits restenosis in a patient in need thereof.

47. A method of monitoring a tumor cell in a patient comprising:
providing a tumor cell from the patient and
incubating the tumor cell with an antibody or fragment thereof of claim 1, thereby staging the tumor cell.

48. The method of claim 47, wherein the method further comprises
determining specific binding of the antibody or fragment thereof relative to a reference standard.

49. The method of claim 47, wherein the antibody or fragment thereof is Y1.

50. A method of isolating a tumor-specific antigen comprising:
obtaining a sample of a cell,
lysing the cell,
identifying a protein ligand of an antibody or fragment thereof of claim 1 and
purifying the protein ligand by passing the cell lysate through an affinity column comprising the antibody or fragment thereof.

51. The method of claim 50, wherein the method further comprises sequencing the protein ligand, thereby identifying the tumor-specific antigen.

52. The method of claim 50, wherein the antibody or fragment thereof is Y1.

53. The method of claim 50, wherein the cells are obtained from a tumor of a human being.

54. The method of claim 53, wherein the tumor is a solid tumor.

55. The method of claim 54, wherein the tumor is small cell lung carcinoma.

56. The method of claim 53, wherein the tumor is a blood-borne tumor.

57. The method of claim 56, wherein the tumor is a leukemia.

58. A method of diagnosing, prognosing, or staging a disease in a patient comprising
providing a sample comprising a cell from the patient and
determining whether the antibody or fragment thereof of claim 1 binds to the cell of the patient,
thereby indicating that the patient is at risk for or has the disease.

59. The method of claim 58, wherein the method further comprises
determining specific binding of the antibody or fragment thereof and
comparing the specific binding of the antibody or fragment thereof to the cell relative to a reference standard.

60. The method of claim 58, wherein Western blotting is used to determine whether the antibody of fragment thereof of claim 1 binds to the cell of the patient.

61. The method of claim 58, wherein the disease is a cancer.

62. The method of claim 61, wherein the cancer is a solid tumor.

63. The method of claim 62, wherein the cancer is small cell lung carcinoma.

64. The method of claim 61, wherein the cancer is a blood-borne tumor.

65. The method of claim 64, wherein the cancer is a leukemia.

66. The method of claim 58, wherein the antibody or fragment thereof is Y1.

67. A method of purging tumor cells from a patient comprising
providing a sample containing cells from the patient and
incubating the cells from the patient with an antibody or fragment thereof of claim 1.

68. The method of claim 67, wherein the purging occurs ex vivo.

69. A process for forming an anthracycline-agent complex comprising
providing an anthracycline;
reacting an adipic acid with the athracycline;
generating an active ester of the adipic acid—anthracycline; and
reacting the adipic acid—anthracycline with a polypeptide to form an anthracycline—agent complex.

70. The method of claim 69, wherein the anthracycline is the anthracycline is selected from the group consisting of doxorubicin, daunorubicin, idarubicin, detorubicin, carminomycin, epirubicin, esorubicin, morpholinodoxorubicin, morpholinodaunorubicin, and methoxymorpholinyldoxorubicin.

71. The method of claim 69, wherein the polypeptide is an antibody or fragment thereof.

72. A complex produced according to the process of claim 69.

73. The complex of claim 72, wherein the anthracycline is doxorubicin, daunorubicin, idarubicin, detorubicin, carminomycin, epirubicin, esorubicin, morpholinodoxorubicin, morpholinodaunorubicin, and methoxymorpholinyldoxorubicin.

74. The complex of claim 72, wherein the polypeptide is an antibody or fragment thereof.

75. An antibody or fragment thereof that binds to an epitope comprising the sequence YD, wherein YD is located within the motif DXYD or within an acidic environment, and wherein X is any amino acid or the covalent linkage between D and Y, and wherein Y is sulfated.

76. A pharmaceutical composition comprising an antibody or fragment thereof of claim 75 and a pharmaceutically acceptable carrier.

77. A diagnostic, prognostic, or staging kit comprising an antibody or fragment thereof of claim 75 and an imaging agent.

78. The diagnostic, prognostic, or staging kit of claim 77, wherein the imaging agent is a radioisotope.

79. A method of inducing antibody-dependent cell cytotoxicity (ADCC) comprising administering to a patient in need thereof the pharmaceutical composition of claim 76.

80. A method of stimulating a natural killer (NK) cell or a T cell comprising administering to a patient in need thereof the pharmaceutical composition of claim 76.

81. A method of inducing cell death comprising administering to a patient in need thereof an antibody or fragment thereof of claim 75, wherein the antibody or fragment thereof complexed to the agent enters the cell and the antibody or fragment thereof is cleaved from the agent, thereby releasing the agent.

82. A method of treating HIV comprising administering to a patient in need thereof a pharmaceutical composition of claim 76.

83. The method of claim 82, wherein the administration prevents entry of HIV.

84. A method of treating a disease comprising administering to a patient in need thereof a pharmaceutical composition of claim 1 or 76

85. The method of claim 84, wherein the method treats cell rolling in a patient in need thereof.


SEQUENCE LISTING (34 sequences)

SEQ ID NO: 1 - protein, 7 aa, Artificial Sequence
  Description of Artificial Sequence Synthetic peptide
YEYLDYD

SEQ ID NO: 2 - protein, 20 aa, Homo sapiens
VRPEHPAETEYDSLYPEDDL

SEQ ID NO: 3 - protein, 4 aa, Artificial Sequence
  Description of Artificial Sequence Synthetic peptide motif
DXYD

SEQ ID NO: 4 - protein, 8 aa, Artificial Sequence
  Description of Artificial Sequence Synthetic peptide
DLYDYYPE

SEQ ID NO: 5 - protein, 8 aa, Artificial Sequence
  Description of Artificial Sequence Synthetic peptide
DLYDYYPE

SEQ ID NO: 6 - protein, 8 aa, Artificial Sequence
  Description of Artificial Sequence Synthetic peptide
DLYDYYPE

SEQ ID NO: 7 - protein, 8 aa, Artificial Sequence
  Description of Artificial Sequence Synthetic peptide
DLYDYYPE

SEQ ID NO: 8 - protein, 8 aa, Artificial Sequence
  Description of Artificial Sequence Synthetic peptide
DLYDYYPE

SEQ ID NO: 9 - protein, 8 aa, Artificial Sequence
  Description of Artificial Sequence Synthetic peptide
DLYDYYPE

SEQ ID NO: 10 - protein, 8 aa, Artificial Sequence
  Description of Artificial Sequence Synthetic peptide
DLYDYYPE

SEQ ID NO: 11 - protein, 8 aa, Artificial Sequence
  Description of Artificial Sequence Synthetic peptide
DLEDYYPE

SEQ ID NO: 12 - protein, 8 aa, Artificial Sequence
  Description of Artificial Sequence Synthetic peptide
DLYEYYPE

SEQ ID NO: 13 - protein, 8 aa, Artificial Sequence
  Description of Artificial Sequence Synthetic peptide
DLYNYYPE

SEQ ID NO: 14 - protein, 8 aa, Artificial Sequence
  Description of Artificial Sequence Synthetic peptide
DLYAYYPE

SEQ ID NO: 15 - protein, 8 aa, Artificial Sequence
  Description of Artificial Sequence Synthetic peptide
DAYDYYPE

SEQ ID NO: 16 - protein, 8 aa, Artificial Sequence
  Description of Artificial Sequence Synthetic peptide
DLYDFYPE

SEQ ID NO: 17 - protein, 8 aa, Artificial Sequence
  Description of Artificial Sequence Synthetic peptide
DLYDAYPE

SEQ ID NO: 18 - protein, 8 aa, Artificial Sequence
  Description of Artificial Sequence Synthetic peptide
DLYDYYAE

SEQ ID NO: 19 - protein, 8 aa, Artificial Sequence
  Description of Artificial Sequence Synthetic peptide
DLYDYYPA

SEQ ID NO: 20 - protein, 17 aa, Artificial Sequence
  Description of Artificial Sequence Synthetic peptide
QATEYEYLDYDFLPETE

SEQ ID NO: 21 - protein, 4 aa, Artificial Sequence
  Description of Artificial Sequence Synthetic peptide motif
DXYD

SEQ ID NO: 22 - protein, 19 aa, Artificial Sequence
  Description of Artificial Sequence Synthetic peptide
DEGDTDLYDYYPEEDTEGD

SEQ ID NO: 23 - protein, 15 aa, Artificial Sequence
  Description of Artificial Sequence Synthetic peptide
EHPAETEYDSLYPED

SEQ ID NO: 24 - protein, 18 aa, Artificial Sequence
  Description of Artificial Sequence Synthetic peptide
MEANEDYEDYEYDELPAK

SEQ ID NO: 25 - protein, 9 aa, Artificial Sequence
  Description of Artificial Sequence Synthetic peptide
GYYDYDFPL

SEQ ID NO: 26 - protein, 15 aa, Artificial Sequence
  Description of Artificial Sequence Synthetic linker peptide
GGGGSGGGGSGGGGS

SEQ ID NO: 27 - protein, 6 aa, Artificial Sequence
  Description of Artificial Sequence Synthetic 6xHis tag
HHHHHH

SEQ ID NO: 28 - protein, 6 aa, Artificial Sequence
  Description of Artificial Sequence Synthetic peptide tag
HTTPHH

SEQ ID NO: 29 - protein, 7 aa, Artificial Sequence
  Description of Artificial Sequence Synthetic peptide
DLYDYYP

SEQ ID NO: 30 - protein, 7 aa, Artificial Sequence
  Description of Artificial Sequence Synthetic peptide
DLYDYYP

SEQ ID NO: 31 - protein, 7 aa, Artificial Sequence
  Description of Artificial Sequence Synthetic peptide
DLYDYYP

SEQ ID NO: 32 - protein, 7 aa, Artificial Sequence
  Description of Artificial Sequence Synthetic peptide
DLYDYYP

SEQ ID NO: 33 - protein, 7 aa, Artificial Sequence
  Description of Artificial Sequence Synthetic peptide
DLYDYYP

SEQ ID NO: 34 - protein, 7 aa, Artificial Sequence
  Description of Artificial Sequence Synthetic peptide
DLYDYYP